(19) United States
(12) Patent Application Publication
(10) Pub. No.: US 2024/0207507 A1
(43) Pub. Date: Jun. 27, 2024

(54) CONTAINMENT, ACTIVATION, DEPLOYMENT, AND INTEGRATED FUNCTIONS OF FLEXIBLE, SLENDER ELEMENTS

(71) Applicant: INSULET CORPORATION, Acton, MA (US)

(72) Inventor: John BUSSIERE, Littleton, MA (US)

(21) Appl. No.: 18/390,450

(22) Filed: Dec. 20, 2023

Related U.S. Application Data
(60) Provisional application No. 63/434,243, filed on Dec. 21, 2022.

(51) Int. Cl. A61M 5/142; A61M 37/00

(52) U.S. Cl. CPC A61M 5/14248; A61M 37/0069; A61M 2005/14284

(57) ABSTRACT

Exemplary embodiments provide methods, apparatuses, and supporting structures for deploying a flexible, slender element (such as a conduit or subcutaneous sensor) into a user's tissue (such as skin tissue). A first embodiment leverages rotary motion to deploy the element while constraining the element to prevent buckling. A second embodiment leverages linear motion to deploy the flexible, slender element with the assistance of an introducer element to provide support. Other embodiments provide support features; for instance, a sacrificial sleeve may impermanently hold or embrace the slender element to temporarily increase the structural stiffness of the slender element. Another support structure may provide for tensioning the user's tissue, such as by causing the skin to bulge in an area where deployment of a slender element is facilitated. The cannula or sensor itself may also be structured or configured to support deployment.

### CROSS-REFERENCE TO RELATED APPLICATION

This application claims priority to and the benefit of U.S. Provisional Application No. 63/434,243, filed Dec. 21, 2022, the entirety of which is incorporated herein by reference.

### BACKGROUND

Advances in miniaturization have allowed for the creation of body-worn devices capable of administering therapeutics, as well as sensing and reporting physiological characteristics. Examples of such devices include (but are not limited to) devices for dispensing insulin, glucagon-like peptide (GLP-1), a combination of insulin and GLP-1, fertility drugs, white blood cell stimulating drugs, or other medicaments.

Such devices generally deploy a needle, conduit, cannula or other element that connects the body-worn device into the user's tissue (e.g., the skin). Such a deployable element is generally referred to as a “conduit” herein, although it is understood that the term conduit encompasses needles, cannulas, and other similar deployable elements. In order determine how much of the therapeutic to dispense, the device may include a sensor (such as a glucose or ketone sensor), which may be part of the conduit or may be separate from it.

The conduit must be large enough to effectively provide the above-noted functionality, but is preferably as small as possible given those constraints. A smaller conduit generally means less pain for the user, both when the element penetrates the skin and during extended deployments (as might be necessary for an automated insulin delivery, or “AID” device). It also means that the overall device can be made smaller, allowing it to be worn unobtrusively under clothing and improving user comfort while wearing it.

At the same time, the conduit needs to be sufficiently rigid to penetrate the skin without buckling. As these conduits become narrower or more flexible and/or slender, they trend towards becoming insufficiently stiff due to intrinsic material physical properties (e.g., the elastic modulus) and very small physical sizes and geometries (e.g., geometric section stiffness). Thin, flexible, slender elements that naturally have low geometric stiffness are notoriously difficult to deploy to appropriate penetration depths into deformable materials such as human skin tissues. They instead trend towards structural compromise due to axial compressive forces, which can result in undesired transverse displacements (e.g., buckling or deflection, such as by bouncing off the top surface of the skin and failing to penetrate, as when the conduit is inserted at an angle) of the structure under critical load conditions or upon insertion or penetration through human skin, for example.

### BRIEF SUMMARY

Exemplary embodiments provide unique conduit deployment assemblies configured to mitigate deployment failures during penetration events and allow for proper functions of use after initial penetration.

In one aspect, an apparatus includes a deployable element configured to be deployed subcutaneously, a rotary mechanism having an outer circumference around at least a portion of which the deployable element is wrapped, the rotary mechanism configured to rotate the deployable element around the outer circumference, and a base having an opening through which the deployable element is configured to extend, where the deployable element is selectively constrained so that the rotation of the deployable element is converted into a linear motion.

In some embodiments, the rotary mechanism may be a sheave or flywheel assembly.

The apparatus may also include a torsion spring configured to supply energy to rotate the rotary mechanism.

The apparatus may also include a stop plate configured to control an extent to which the deployable element extends beyond the opening.

The apparatus may also include a fluid conduit configured to connect the deployable element to a reservoir.

The use of a rotational mechanism as described above may allow the apparatus to be effective while still being relatively small (i.e., less than 0.5″×0.5″×0.375″).

The deployable element may be selected from the group consisting of a cannula, a conduit, a needle, or a sensor. In some embodiments, the deployable element is a glucose sensor configured to perform continuous glucose monitoring, or a ketone sensor.

The apparatus may also include at least one housing configured to enclose the apparatus. In some embodiments, the apparatus deploys a sensor and the at least one housing encloses only the apparatus. In other embodiments, the apparatus may be a part of an automated insulin delivery (AID) device, and the at least one housing encloses both the apparatus and the AID device.

The apparatus may also include at least one convex or concave protrusion provided on the base, where the protrusion is sized and shaped to cause a user's skin to be tensioned in the vicinity of the opening when the base is pushed against or adhered to the skin.

In some embodiments, the deployable element has a circular cross-section (such as a laminated or coated wire). In others, the deployable element may have a rectangular cross-section (as might be the case in a conduit). In some embodiments, the deployable element has a laminated composition of includes at least one polymer, at least one metal, and a coating. In some embodiments, the deployable element includes one or more electrically conductive signal traces connected to embedded electronics hardware.

In another aspect, an apparatus includes a deployable element extending linearly in a first direction and configured to be deployed subcutaneously, an introducer extending in the first direction parallel to the deployable element, a base having an opening through which the deployable element is configured to extend, and a linear deployment mechanism configured to cause the deployable element and the introducer to extend in the first direction.

The introducer may include a geometric interface having an angled structure, and further includes a suspension having a suspension opening through which the deployable element and the introducer pass, where suspension opening is shaped so that the deployable element is permitted to continue upon its original linear trajectory until an established stop point is reached and the introducer is forced by the angled structure through a portion of the suspension opening that releases the introducer from a driving force of the linear deployment mechanism.

The apparatus may also include an extension spring connected to an end of the introducer, where the extension spring is configured to reverse a direction of the introducer's displacement to secure a sharpened end of the introducer within a protected structure.

The apparatus may also include a sacrificial sleeve joining the deployable element with the introducer.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A is a perspective view an exemplary rotary cannula deployment mechanism according to a first embodiment.

FIG. 1B is a side view an exemplary rotary cannula deployment mechanism according to a first embodiment.

FIG. 1C is a perspective view of an exemplary rotary cannula deployment mechanism according to a second embodiment.

FIG. 1D depicts an exemplary dampener suitable for use with exemplary embodiments.

FIG. 1E depicts an internal view of an exemplary dampener suitable for use with exemplary embodiments.

FIG. 1F is a side view of an exemplary rotary cannula deployment mechanism with a dampener.

FIG. 2 is a perspective view depicting the sheave assembly in a first, undeployed state.

FIG. 3 is a perspective view depicting the sheave assembly in a second, deployed state.

FIG. 4A-FIG. 4D provide various closeup views of the sheave assembly of the first embodiment.

FIG. 5 provides various views of the deployment mechanism of the first embodiment enclosed in a standalone housing.

FIG. 6 provides various views of the deployment mechanism of the first embodiment enclosed in a housing capable of accommodating other components, such as an automated insulin delivery (AID) device.

FIG. 7A is a perspective exploded view of an exemplary rotary cannula deployment mechanism according to a first embodiment.

FIG. 7B depicts a perspective view from the opposite side as compared to FIG. 8A.

FIG. 8 is a side view depicting the deployable element 102 of the first embodiment in more detail.

FIG. 9 is a side view depicting an alternate configuration for the deployable element 102 of the first embodiment.

FIG. 10A is a front and side perspective view depicting the sheave assembly of the first embodiment in more detail.

FIG. 10B is a side and rear perspective view depicting the sheave assembly of the first embodiment in more detail.

FIG. 11 is a perspective view showing electrical connections between the sensor and onboard front-end electronics, and an electrical service loop for connecting the front-end electronics to back-end electronics in a drug delivery device main body.

FIGS. 12A-12D depict an alternative release mechanism suitable for use with exemplary embodiments.

FIG. 13 is a perspective exploded view of an exemplary linear cannula deployment mechanism according to a second embodiment.

FIG. 14 is a side- and bottom-perspective view of the assembled linear cannula deployment mechanism of the second embodiment in a first, undeployed state.

FIG. 15 is a side- and bottom-perspective view of the assembled linear cannula deployment mechanism of the second embodiment in a second, deployed state.

FIG. 16 depicts the introducer of the second embodiment in more detail.

FIG. 17 is a side view of a structure for tensioning a user's epidermis suitable for use with any of the described embodiments.

FIG. 18 is a side view of an alternative epidermal tensioning structure suitable for use with any of the described embodiments.

FIG. 19 is a perspective view of an epidermal tensioning structure.

FIG. 20 is a side view of the epidermal tensioning structure of FIG. 17 showing different possible protrusion profile shapes.

FIG. 21A is a perspective view depicting an exemplary cannula structure suitable for use with any of the described embodiments.

FIG. 21B illustrates an aspect of the subject matter in accordance with one embodiment.

FIG. 21C illustrates an aspect of the subject matter in accordance with one embodiment.

FIG. 22 is a side view of the exemplary cannula structure of FIG. 21A.

FIG. 23 is a perspective view depicting an exemplary sensor structure suitable for use with any of the described embodiments.

FIG. 24A depicts an exemplary rotary cannula deployment mechanism that utilizes an introducer to penetrate the outermost layers of skin

FIG. 24B depicts the rotary cannula deployment mechanism of FIG. 24A with an exemplary cowling.

FIGS. 25A-B depict an exemplary introducer suitable for use with the rotary cannula deployment mechanism of FIG. 24A.

FIGS. 26A-C depict exemplary introducers according to various embodiments.

FIG. 27A shows how the introducer is positioned in reference to cowling features in an exemplary rotary cannula deployment mechanism.

FIGS. 27B-27C provide various views of a sheave for the rotary cannula deployment mechanism.

FIGS. 28A-28D depict an exemplary retaining block for use with a rotary cannula deployment mechanism in various stages of assembly.

FIGS. 29A-C depict the rotary cannula deployment mechanism with the introducer moving through various stages of deployment and retraction.

FIGS. 30A-30B depict the rotary cannula deployment mechanism in a pre-deployment and fully-penetrating state, respectively.

FIG. 31 depicts the interface between the introducer and the spring, according to an exemplary embodiment.

FIG. 32A is a side-view showing the rotary cannula deployment mechanism in a pre-deployment state.

FIG. 32B is a side-view showing the rotary cannula deployment mechanism in a fully-penetrating state.

### DETAILED DESCRIPTION

Exemplary embodiments provide methods, apparatuses, and supporting structures for deploying a flexible, slender element (such as a conduit or subcutaneous sensor) into a user's tissue (such as skin tissue).

A first embodiment leverages rotary motion to deploy the element while constraining the element to prevent buckling. The constraints may allow the element to be deployed without the assistance of an introducer element that supports the flexible element.

Utilizing rotary motion derived from a source of harnessed potential energy (e.g., a torsion spring) and leveraging the moment of a force applied to a selectively constrained thin element at the radius of a flywheel or sheave, the thin element is guided with minimum frictional resistance in a circumferential-to-linear motion and forced into the skin tissue layers at the distal sharpened end of the slender element. Such circumferential wrapping and initial containment provide for a neatly packaged (minimal spatial volume of mechanism) deployment method of sensor elements to depths of subcutaneous tissue below the outer surface of skin (in some embodiments, about a 4-7 mm depth).

A second embodiment leverages linear motion to deploy the flexible, slender element with the assistance of an introducer element to provide support. Utilizing linear motion derived from a source of harnessed potential energy (e.g., a helical compression spring) and leveraging of a force applied to selectively constrained configuration of a single or multiple thin elements, the thin element(s) is guided with minimum frictional resistance in a linear motion and forced into the skin tissue layers initiated at the distal sharpened end of the slender element (a subcutaneous introducer in this embodiment). Such linear containment provides for a neatly packaged (minimal spatial volume of mechanism) deployment method of sensor elements to depths of subcutaneous tissue below the outer surface of skin (4-7 mm).

Other embodiments provide support features that improve the functionality of the above-mentioned embodiments. For instance, a sacrificial sleeve may impermanently hold or embrace the slender element to temporarily increase the structural stiffness of the slender element.

Another support structure may provide for tensioning the user's tissue, such as by causing the skin to bulge in an area where deployment of a slender element is facilitated. This structure may include a protruding annulus structure on a body worn or applied device in contact, and/or adhered, with the skin during insertion of the slender element being deployed into the human skin layers.

The cannula or sensor itself may also be structured or configured to support deployment. For instance, laminated composition of polymers, metals, and other materials may be combined into varied cross-sectional shapes. These elements may be employed to develop desired directional stiffness for improved deployment penetration and/or to maintain electrically conductive signal conduit/traces to embedded electronics hardware.

### Exemplary Embodiments

As an aid to understanding, a series of examples will first be presented before detailed descriptions of the underlying implementations are described. It is noted that these examples are intended to be illustrative only and that the present invention is not limited to the embodiments shown.

Reference is now made to the drawings, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form in order to facilitate a description thereof. The intention is to cover all modifications, equivalents, and alternatives consistent with the claimed subject matter.

In the Figures and the accompanying description, the designations “a” and “b” and “c” (and similar designators) are intended to be variables representing any positive integer. Thus, for example, if an implementation sets a value for a=5, then a complete set of components 122 illustrated as components 122-1 through 122-a may include components 122-1, 122-2, 122-3, 122-4, and 122-5. The embodiments are not limited in this context.

### Introducer-Less Rotary Cannula Deployment Mechanism

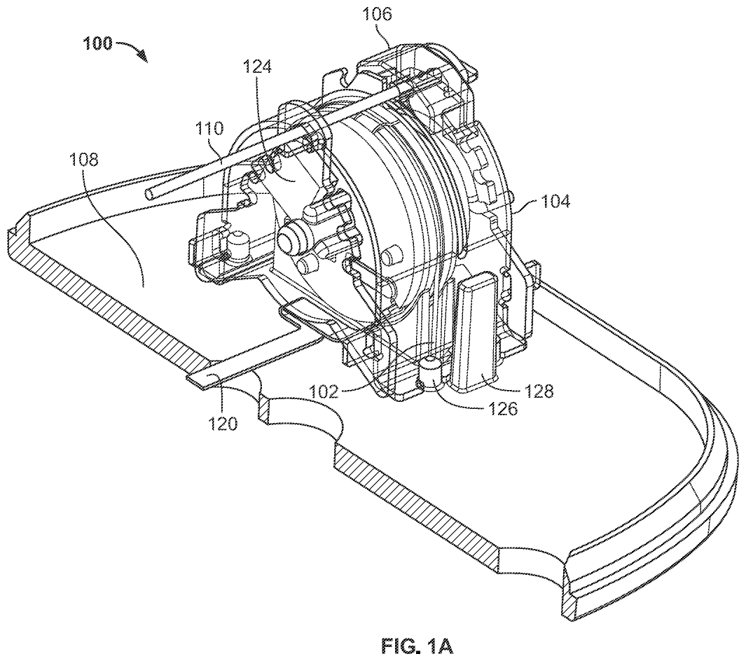

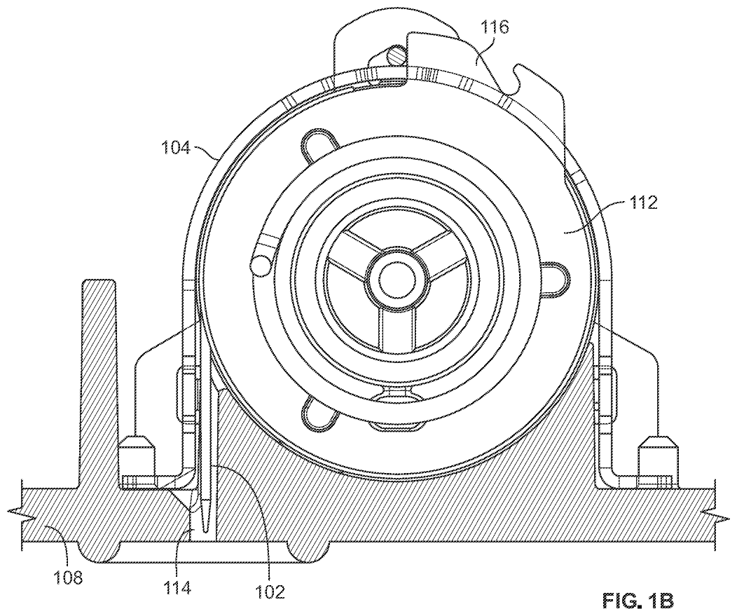

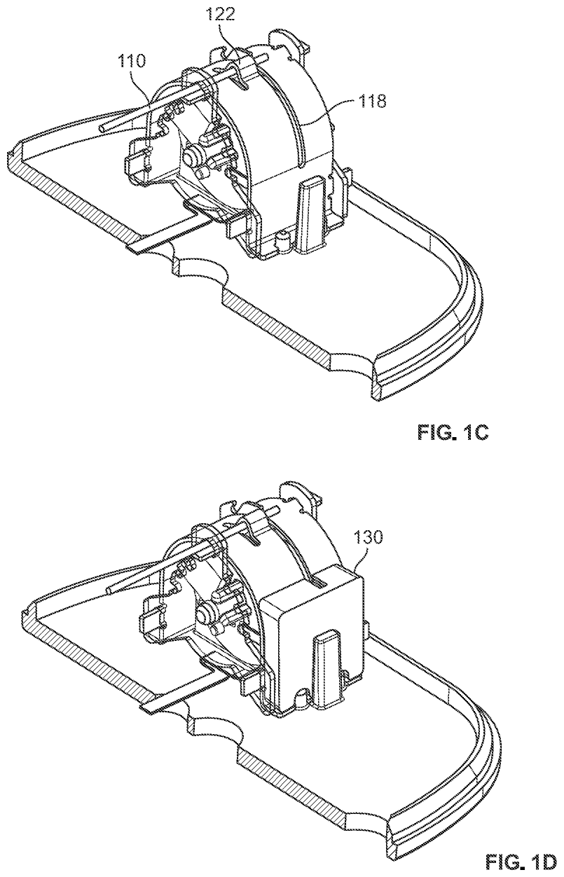

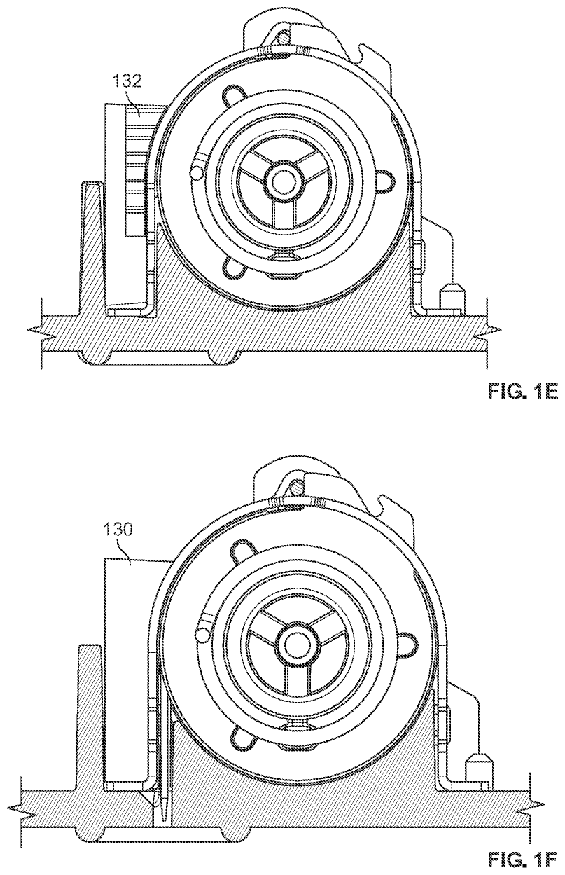

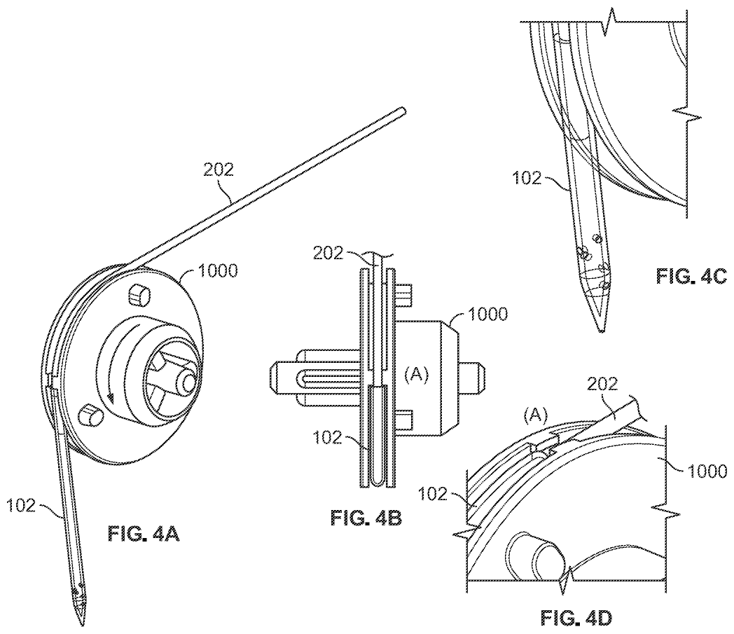

A first embodiment provides an introducer-less rotary conduit deployment assembly 100, as shown in FIG. 1A—FIG. 4D. The conduit deployment assembly 100 may be used to deploy a conduit, a sensor, or both (as appropriate).

FIG. 1A and FIG. 1B depict the assembled conduit deployment assembly 100. The conduit deployment assembly 100 comprises a deployable element 102, which may be a cannula, needle, conduit, sensor, etc. For instance, the deployable element 102 may be a cannula or needle for delivering a medicament such as insulin. The deployable element 102 may be a glucose sensor or ketone sensor (e.g., an electrochemical sensor; a fiber optic sensor, a wire sensor, an optofluorescence sensor, etc.), a temperature sensor, or an activity sensor. In some embodiments, multiple sensors may be combined into a single deployable element 102, as might be the case where one side of the deployable element 102 serves as a glucose sensor and the opposite side serves as a ketone sensor.

In this example, the deployable element 102 rotates along the outer radius of a flywheel or sheave until the rotational motion is converted to linear motion and the deployable element 102 moves linearly into or out of an opening 114 in a base 108 that supports the conduit deployment assembly 100 (e.g., on the skin of the user). The deployable element 102 may be selectively constrained by the cowling 104 that surrounds the deployable element 102 and sheave. Additionally or alternatively, the cowling 104 may take the form of a housing for the conduit deployment assembly 100.

The conduit deployment assembly 100 may be positioned and/or retained on the base 108 using one or more base protrusions 126 and/or ribs 128 that fit into corresponding cutouts in the bottom of the cowling 104, which may be flared outwards to allow for a connection to be made between the conduit deployment assembly 100 and the base 108.

Utilizing rotary motion derived from a source of harnessed potential energy (in this embodiment a torsion spring controlled by a release 110 fitting into a bearing block 106) and leveraging the moment of a force applied to the deployable element 102 at the radius of the sheave subopenings 804b, the deployable element 102 is guided with minimum frictional resistance in a circumferential to linear motion and forced into the skin tissue layers at the distal sharpened end of the deployable element 102 (a subcutaneous sensor in this application, but alternatives may be used as explained above and below). Such circumferential wrapping and initial containment allow for a mechanism with a relatively small spatial volume (e.g., less than about 0.25″×0.25″×0.375″).

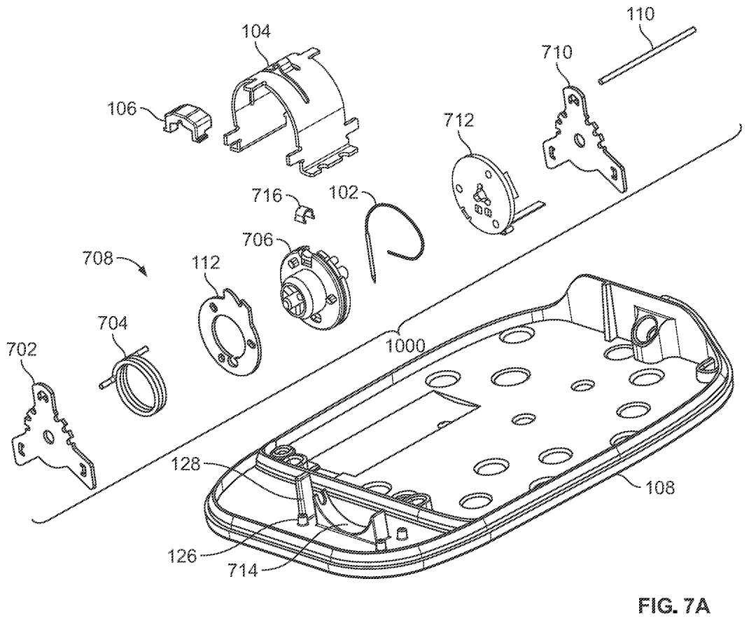

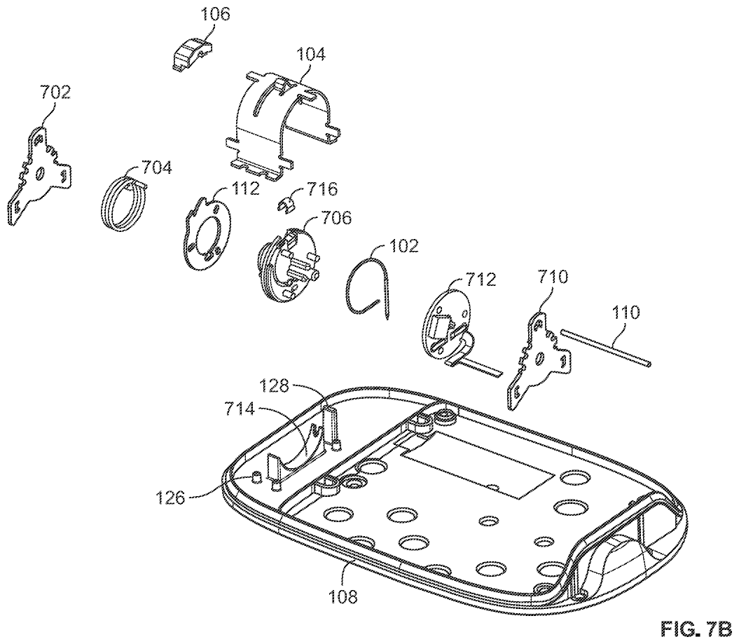

The conduit deployment assembly 100 allows a sensor or other elements to be deployed to depths of subcutaneous tissue below the outer surface of skin (e.g., ˜4-7 mm). The depth of penetration may be controlled by the shape of a stop plate 112 mounted to the sheave. As shown in FIG. 1B, FIG. 1C, FIG. 7A and FIG. 7B, the stop plate 112 includes a protrusion 116 that fits into a groove 118 in the cowling 104, and further includes openings allowing the stop plate 112 to be mounted to the sheave. When the sheave turns, the stop plate 112 rotates and the protrusion 116 moves within the groove 118 until hitting a stopping point at the end of the groove 118, which prevents the sheave from rotating further (and therefore stops the linear motion of the deployable element 102).

In the case where the deployable element 102 is a sensor, the sensor may relay signals back to integrated front-end electronics 124 on board the conduit deployment assembly 100. The integrated front-end electronics 124 may include a processor, which may be a relatively less powerful processor as compared to a processor on board a larger body-worn device that the conduit deployment assembly 100 is a part of or attached to. Some limited front-end-processing may be performed on the sensor signals by the integrated front-end electronics 124. The resulting data may be transmitted via a service portion 120 to the processor on the body-worn device.

In the embodiments of FIG. 1A and FIG. 1B, the release 110 is secured in a bearing block 106 until it is pulled away from the stop plate 112. FIG. 1C depicts an alternative configuration where the release 110 is secured in a cowling support 122 formed in the cowling 104. The cowling support 122 may simply be a raised loop formed from the material of the cowling 104, or may be a separate element attached to the cowling 104.

FIG. 1D-FIG. 1F depict an optional damper 130 suitable for use with exemplary embodiments. For example, the damper 130 may be a viscoelectric damper attached to the cowling 104 and filled with a viscous medium to absorb energy from the conduit deployment assembly 100. The damper 130 may include one or more ridges 132 or feathered elements that the 102 may wrap through, where the size and shape of the ridges 132 configured to slow down the motion of the spring that deploys the deployable element 102. In some embodiments, the ridges 132 may be set in a configuration to act only on a part of the stroke that deploys the deployable element 102 (such as the last ½-¼ of the stroke).

FIG. 1F also shows an exemplary structure for the deployable element 102. As shown, an outer tubular structure (e.g., a thin-walled extruded polymer) may surround an inner core that includes a sensor element. The tubular structure may be completely straight or may have a flared (or other shaped) end geometry for integration. This tubular structure may provide additional protection to the outer most coatings of the deployable element 102 as the rotation of the sheave drives the linear (down-ward) excursion of the sensor relative to the static housing and cowling components. This tubular sleeve may remain motionless as the sensor travels through the inner diameter of the tube. Accordingly, a low friction polymer may be a suitable material choice.

Furthermore, there may optionally be a low radial force/compression seal provided at the interface of the housing enclosure and the exiting location of the deployable element 102 (i.e., the opening 114) where it interfaces with the body. This seal may be a low durometer silicone polymer (or compatible elastomer) that could be insert molded to the housing base, or it could be a small o-ring shape, or a more sophisticated “duck-billed” or “conical funnel” type geometry. Such a seal may protect against liquid ingress at that interface.

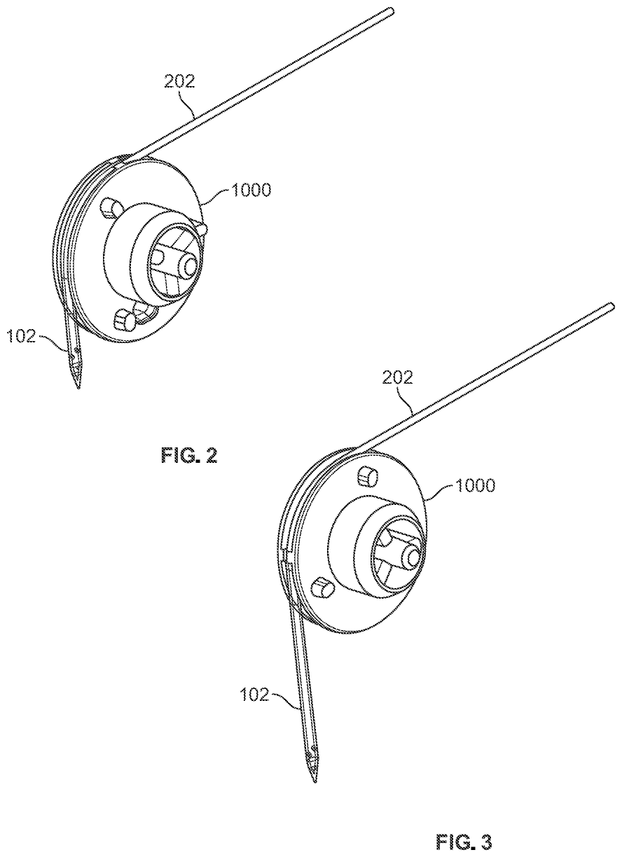

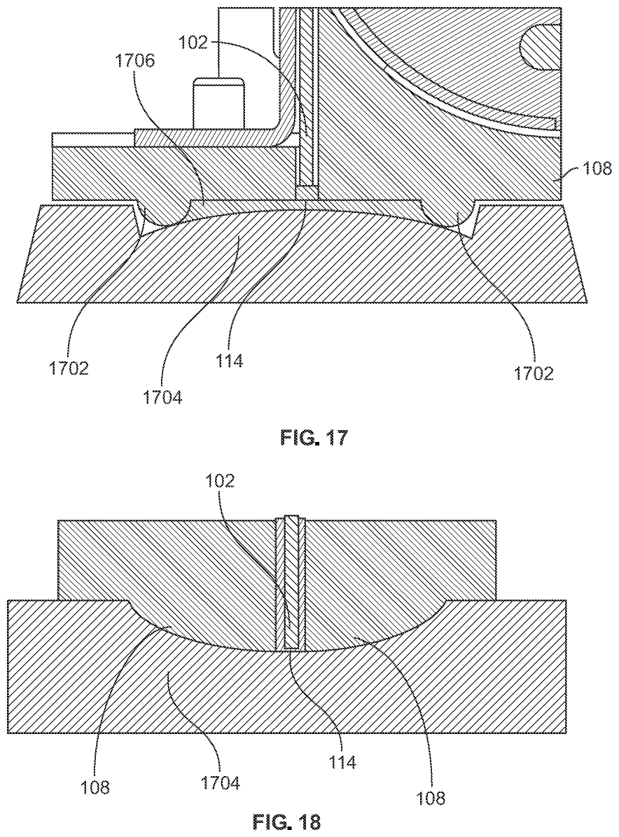

FIG. 2, FIG. 3, and FIG. 4A—FIG. 4D show the deployment of the deployable element 102. In an initial state (FIG. 2 and FIG. 4C), the deployable element 102 is undeployed and withdrawn through the opening 114 (shown, for example, in FIG. 17). As the sheave assembly 1000 rotates, it drives the deployable element 102 in a linear motion (downward, in FIG. 2, FIG. 3, and FIG. 4A) until the deployable element 102 reaches its fully deployed extent (FIG. 3 and FIG. 4A).

FIG. 2, FIG. 3, FIG. 4A, FIG. 4B, and FIG. 4D further show an optional fluid conduit 202, which may be used when the deployable element 102 represents a cannula, conduit, or needle for delivering a drug (such as liquid insulin). The fluid conduit 202 connects to the deployable element 102 (e.g., through a flexible, elastic, tapered, or extensible portion that allows the interface between the deployable element 102 and fluid conduit 202 to move as the sheave rotates, or by moving the length of the fluid conduit 202 into/out of an attached reservoir). The fluid conduit 202 may connect to a reservoir and allow fluid to be delivered from the reservoir through the deployable element 102. The fluid conduit 202 may form a bend when the deployable element 102 is in its undeployed or initial state (FIG. 2 and FIG. 4C) and this bend may have the necessary slack to allow the deployable element 102 to deploy without interrupting fluid connections from the reservoir to the fluid conduit 202 to the deployable element 102. The deployable element 102 may have a sharpened distal end, an open lumen at its distal end fluidly connected to fluid conduit 202, and/or one or more holes proximate to its distal end (e.g., as shown in FIG. 4C) for delivering a medicament such as insulin.

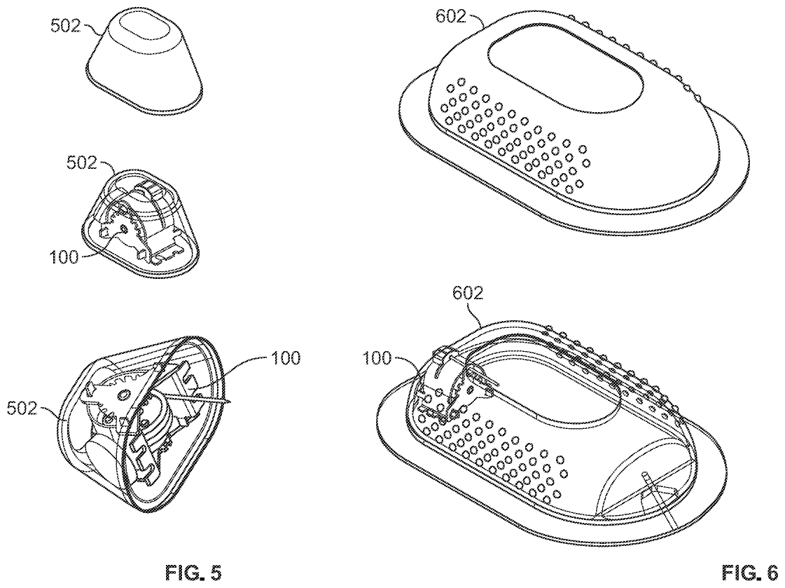

Due to the use of rotary motion to drive the deployable element 102, the conduit deployment assembly 100 can be held to such a small size. Accordingly, it can be deployed in a relatively small housing, either on its own (FIG. 5) or together with other elements of a drug-delivery device (e.g., an automated insulin delivery, or “AID,” device; see FIG. 6).

In the standalone example of FIG. 5, the conduit deployment assembly 100 fits within its own conduit assembly housing 502. In this form factor, the conduit deployment assembly 100 could be used, for example, to deploy a standalone sensor (such as a glucose sensor for a continuous glucose monitor).

In the combined example of FIG. 6, the conduit deployment assembly 100 is contained within a larger unitary device housing 602 that may also house other components of a drug-delivery device. For example, the conduit deployment assembly 100 may be used to deploy a glucose sensor or ketone sensor (as in FIG. 5) that is used to control the delivery of metered amounts of insulin from an AID device. The AID device may also be contained within the unitary device housing 602. Separately or together with the glucose monitor, the AID device may also make use of a conduit deployment assembly 100 to deploy a needle, cannula, or conduit for delivering the insulin or other medicament.

FIG. 7A-FIG. 7B depict exploded views of the various components of the conduit deployment assembly 100 from different perspectives.

As previously noted, the conduit deployment assembly 100 sits on a base 108 that is shaped to hold a sheave assembly 1000. In particular, the base 108 includes a semicircular rest configured to support a sheave assembly 1000.

The sheave assembly 1000 may be controlled by a release 110, which includes a curved portion that fits within a bearing block 106. The bearing block 106 connects to a first bearing plate 702 that is fixed to a first end of the cowling 104, thereby holding the bearing block 106 in place. The release 110 restrains the protrusion 116 of the stop plate 112. When the release 110 is pulled (see FIG. 1A), the curved portion is pulled out of the bearing block 106 into the vicinity of the protrusion 116, thereby releasing the protrusion 116 (and, by extension the stop plate 112) from being restrained by the release 110.

The release 110 (in this and the second embodiment) may be activated by a shape memory alloy (SMA) wire that changes length in response to an outside stimulus (e.g., an application of an electric current or a change in temperature). The SMA wire may be activated by circuitry that, for example, responds to a transmitter/receiver in the conduit deployment assembly 100 indicating that it is time to deploy the deployable element 102 (as might be the case if the conduit deployment assembly 100 is part of a drug delivery device controlled, e.g., by a signal from a mobile phone or similar device), by a button press, etc. Alternatively, the release 110 may be manually triggered by a user, such as by pushing a button at an outside surface of the unitary device housing 602 (such as a housing of an insulin pump) or pulling a prong or the release 110 itself, which may be accessible to a user at an outside surface of unitary device housing 602. In this latter example, release 110 may be removed entirely by a user while triggering insertion of deployable element 102, and may be discarded by the user. In an alternative embodiment, release 110 may be connected to a gearing mechanism and/or a plunger element in a reservoir of a drug delivery device that is driven by a gearing mechanism. As the gearing mechanism advances, it may reach a threshold point where the release 110 is pulled or pushed so as to cause deployment of the deployable element 102.

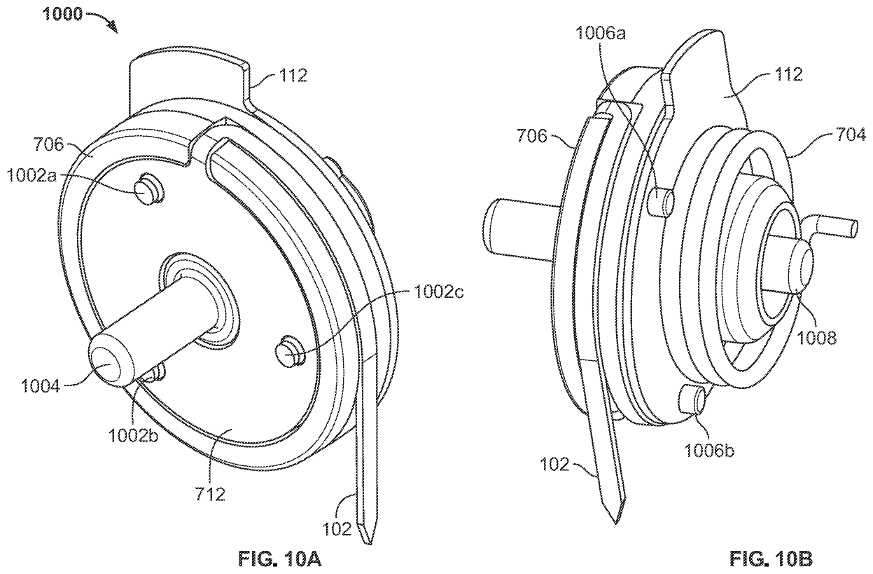

The stop plate 112 is fixed to a sheave 706 (see FIG. 10A and FIG. 10B for details) that receives energy from a spring 704 (in this example, a torsion or coil spring). When the stop plate 112 is released, the sheave 706 turns under the action of the spring 704, causing the protrusion 116 to rotate inside a groove in the cowling 104. When the protrusion 116 reaches the end of the groove, the stop plate 112 is restrained and ceases moving.

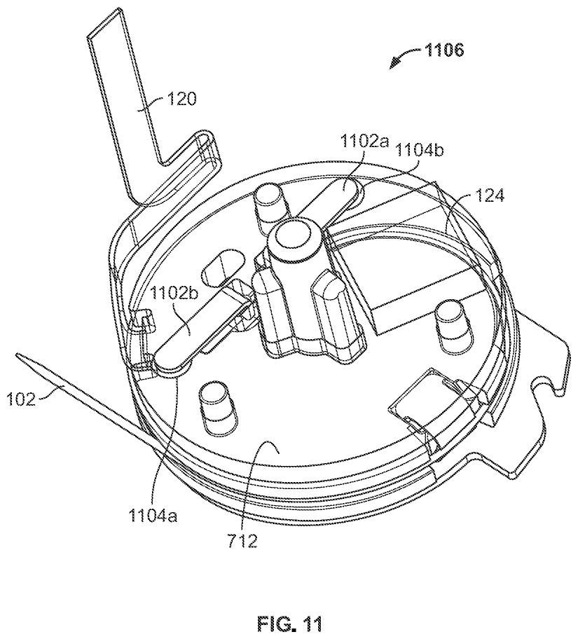

A deployable element support 712 is also mounted to the sheave 706. The deployable element support 712 includes a deployable element 102 (in this case, a sensor in the form of a laminated wire), which is wrapped circumferentially around the sheave 706 and optionally inserted into a ridge in the deployable element support 712, as shown in FIG. 11). The deployable element 102 is further constrained by the cowling 104 so that it rotates within a relatively small passage inside the conduit deployment assembly 100. For example, the passage may be approximately 40 thousandths of an inch wide when deploying a sensor in the form of a wire that has a diameter of 4 thousandths of an inch.

As shown in the example of FIG. 1B, the structures of the cowling 104 and base 108 control the shape of this passage. The passage is configured so that the rotational motion of the deployable element 102 is converted into linear motion in the vicinity of an opening in the base 108. The opening is positioned to receive the end of the deployable element 102 as it moves linearly towards a user's skin.

The sheave 706 is mounted to a second end of the cowling 104 through a second bearing plate 710. The sheave 706 may be properly positioned with respect to the bearing plate 710 using a spacer 708.

The conduit deployment assembly 100 may be mounted to a support 714 that may form a part of a housing or serve as a cradle or tray for the drug delivery device (e.g., unitary device housing 602) or glucose sensing device.

FIG. 8 depicts the deployable element support 712 in more detail.

The deployable element support 712 provides support for the deployable element 102 and connects the deployable element 102 to the sheave 706, properly positioning the deployable element 102 around the outer circumference of the sheave 706.

The deployable element support 712 includes a main body 802 that is circular in shape (to correspond to the shape and general size of the sheave 706). It also includes a sheave main opening 806 sized and shaped to receive a main support pillar extending from the sheave. For further support and to properly orient the deployable element support 712, the main body 802 also includes sheave subopening 804a, 804b, and 804c. These subopenings correspond to sub-supports on the sheave 706.

The deployable element support 712 may also include circuitry 808, such as embedded electronic hardware used to collect data from a sensor or control the flow of a liquid drug into a cannula. In some embodiments, the deployable element 102 may include conductive traces that connect (e.g.) a sensor through the deployable element 102, into the deployable element support 712 and the associated circuitry 808.

In one example, the deployable element support 712, the service portion 120, and/or the deployable element 102 may be formed from a multilayer circuit board, such as a printed circuit board (PCB). One example of the makeup of the different layers of the circuit board is shown below in Table 1:

TABLE 1

| Thickness (inches) | Material | Purpose |
|---|---|---|
| 0.0005 | Polyimide | Coverlay |
| 0.0005 | Adhesive | Coverlay |
| 0.00035 | Copper | Copper Clad |
| 0.001 | Polyimide | Copper Clad |
| 0.0005 | Adhesive | Adhesive |
| 0.00035 | Copper | Copper Clad |
| 0.001 | Polyimide | Copper Clad |
| 0.00035 | Copper | Copper Clad |
| 0.005 | Adhesive | Adhesive |
| 0.001 | Polyimide | Copper Clad |
| 0.00035 | Copper | Copper Clad |
| 0.0005 | Adhesive | Coverlay |
| 0.0005 | Polyimide | Coverlay |
| 0.0005 | Adhesive | Adhesive |
| 0.001 | Stainless Steel | Stiffner |
| 0.001 | Adhesive | Coverlay |
| 0.0005 | Polyimide | Coverlay |

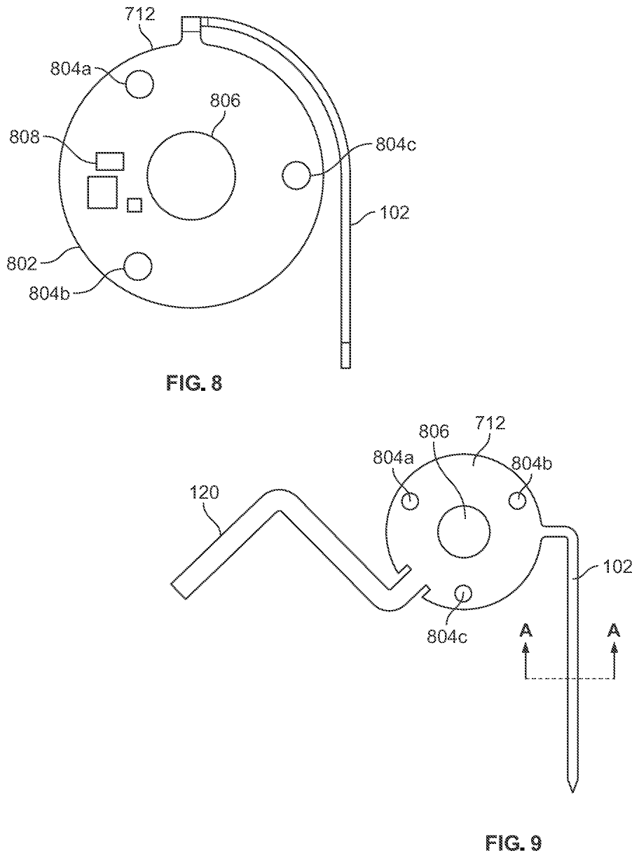

FIG. 9 depicts an alternative embodiment for the deployable element support 712. In addition to the previously-described elements, the deployable element support 712 of FIG. 9 also includes a service portion 120.

FIG. 10A and FIG. 10B depict the sheave assembly 1000 in more detail.

In the sheave assembly 1000, the sheave 706 connects to the deployable element support 712 through a set of sheave supports that pass through corresponding openings in the deployable element support 712. A sheave main front support 1004 passes through the sheave main opening 806 in the deployable element support 712, while sheave front sub-support 1002a, 1002b, 1002c pass through sheave subopenings 804a, 804b, 804c. The sheave main front support 1004 then passes through the spacer 708 and is received in a corresponding opening in the bearing plate 710, which supports the sheave 706.

On the opposite side, similarly situated sub-supports pass through corresponding openings in the stop plate 112. A sheave main rear support 1008 is surrounded by the spring 704, which includes a bent end that connects through the openings in the stop plate 112 and sheave 706 to impart energy to the sheave 706. The sheave main rear support 1008 then passes into a corresponding opening in the bearing plate 702, which supports the sheave 706.

FIG. 11 is a perspective view showing electrical connections between a sensor that is contained in (or forms) the deployable element 102 and integrated front-end electronics 124, and an electrical service portion 120 for connecting the integrated front-end electronics 124 to back-end electronics in a drug delivery device main body.

The sensor in the deployable element 102 may be electrically connected to the deployable element support 712, and may transmit electrical sensor signals to one or more contact points 1104a, 1104b on the deployable element support 712. One or more electrically conductive contacts 1102a, 1102b may be attached to the contact points 1104a, 1104b, respectively, to transmit the signals to the integrated front-end electronics 124. After initial processing, the integrated front-end electronics 124 may transmit processed data to a main processor of the body-worn device via circuitry in the service portion 120. The service portion 120 may connect to circuitry on the body-worn device for the purpose of transferring the processed data (and/or, optionally, raw sensor signals).

This configuration allows data to be initially processed as close to the sensor as possible, which improves computation times and reduces the workload on the main processor (back-end electronics) of the body-worn device.

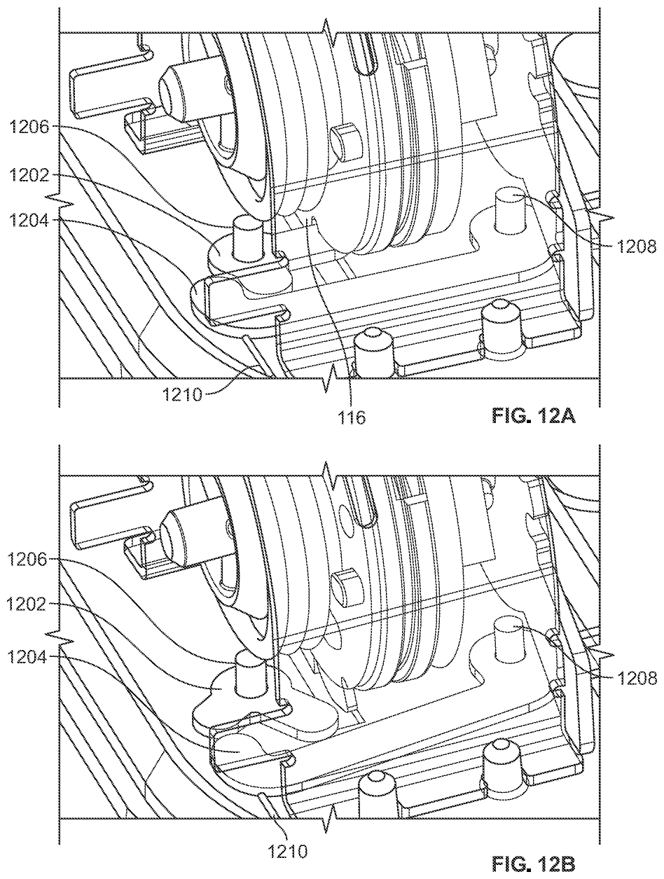

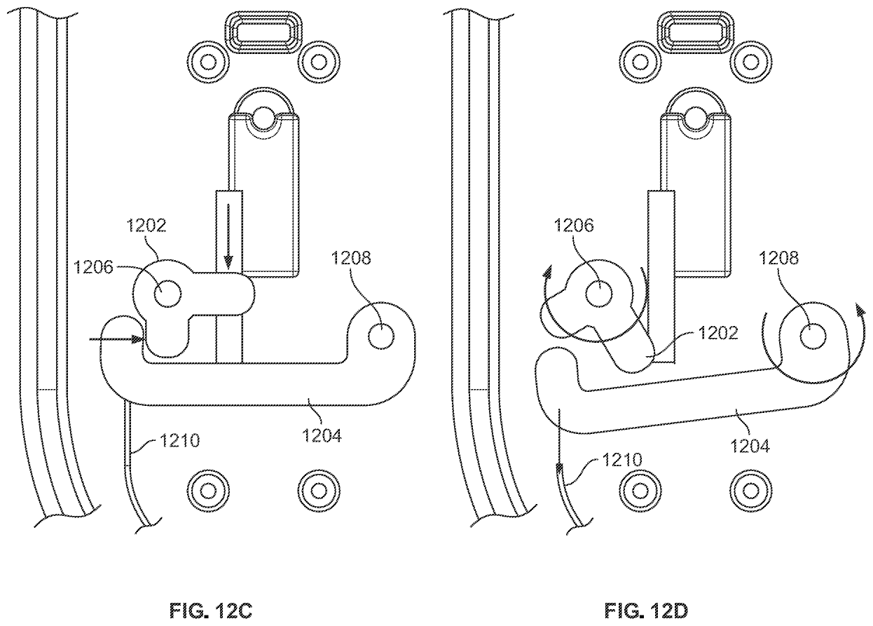

The embodiments described above utilize a release 110 that can be pulled to release the protrusion 116 on the stop plate 112, which allows the sheave 706 to rotate due to the action of the spring. In these embodiments, the release 110 serves to hold the stop plate 112 in place until activated. FIGS. 12A-12D depict an alternative release mechanism suitable for use with exemplary embodiments, which may be useful when space is limited.

In this release mechanism, the protrusion 116 on the stop plate 112 is held in place by two pins mounted to the base 108. A first pin 1202 is L-shaped and mounted to a first pin pivot 1206. The first pin 1202 may be configured to hold the protrusion 116 in place until it swivels on the first pin pivot 1206 to move the first pin 1202 out of the way of the protrusion 116.

To that end, a second pin 1204 is C-shaped and mounted to a second pin pivot 1208 at one end. At the other end, the second pin 1204 is attached to a release trigger 1210, such as an SMA wire.

Before the trigger 1210 is activated, the release mechanism is in the configuration shown in FIG. 12A and FIG. 12C. In this configuration, the protrusion 116 pushes on the first pin 1202 (indicated by the down arrow in FIG. 12C). The force imparted by the protrusion 116 would cause the first pin 1202 to rotate on the first pin pivot 1206, except that the first pin 1202 is prevented from rotating by the second pin 1204.

When the trigger 1210 is activated (e.g., by manually pulling on the trigger 1210 or by applying a current to the trigger 1210 when the trigger 1210 is embodied as an SMA wire). This causes the second pin 1204 to rotate around the second pin pivot 1208 (FIG. 12D), which releases the first pin 1202. The first pin 1202 is now free to rotate round the first pin pivot 1206, thus moving the first pin 1202 away from the protrusion 116. Without the impediment of the first pin 1202, the sheave 706 is free to rotate under the action of the spring 704.

Linear Deployment Mechanism with an Optional Introducer

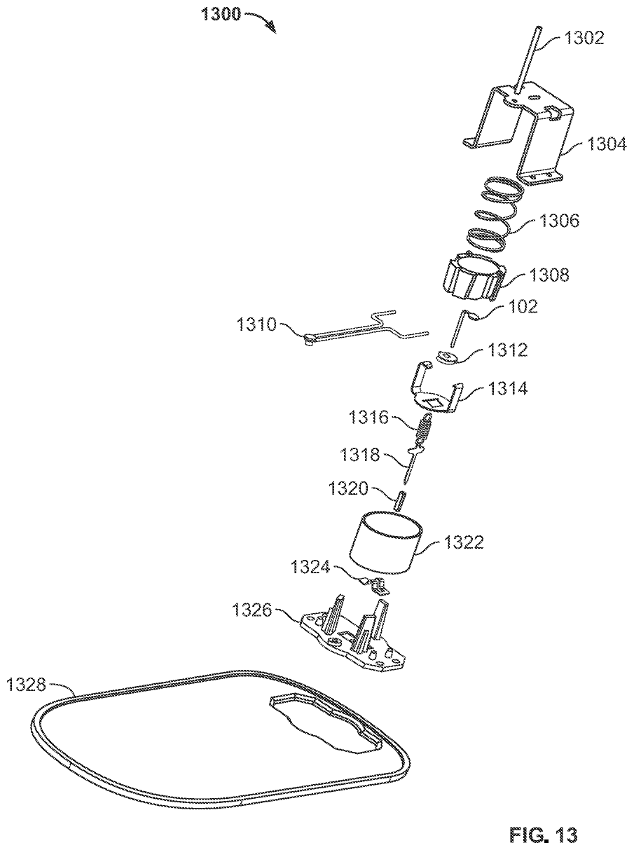

An alternative to the first embodiment utilizes linear, rather than rotary, movement. Examples of these embodiments are shown in FIG. 13-FIG. 16.

In this embodiment, the deployable element 102 is not constrained by the cowling and base, and might therefore be more prone to buckling than the first embodiment. Accordingly, in order to stiffen the deployable element 102, an optional introducer 1318 is provided. The introducer 1318 may be placed parallel to the deployable element 102 so that the introducer 1318 supports the deployable element 102 during deployment.

However, it may be undesirable to leave the introducer 1318 embedded in the user's skin for the length of time that the deployable element 102 is deployed. This may increase the size of the entry point into the user's skin, causing discomfort (among other disadvantages). Accordingly, exemplary embodiments provide a design that allows the introducer 1318 to be automatically retracted after it has served the purpose of assisting in deploying the deployable element 102.

FIG. 13 is an exploded view showing the various parts of a linear conduit deployment assembly 1300.

An anti-rotation pin 1302 connects a harness 1304 to the housing holding the conduit deployment assembly 1300. The anti-rotation pin 1302 prevents the harness 1304 from rotating (e.g., due to a force imparted by a deployment spring 1306). The harness 1304 may connect, at the base of its legs, to a base 1326. The base may be sized and shaped complimentary to an opening in a base 1326 that forms the bottom of the housing in which the conduit deployment assembly 1300 is contained.

The harness 1304 may be configured to support other elements of the conduit deployment assembly 1300, including a deployment spring 1306, bearing cup 1308, deployable element 102, buffer plate 1312, and suspension 1314.

The deployment spring 1306 (e.g., a helical compression spring) stores and releases energy which allows the deployable element 102 to be driven linearly into the user's skin. The deployment spring 1306 pushes against a bearing cup 1308 that is sized and shaped to press against the deployable element 102, thereby transferring the energy from the deployment spring 1306 to the deployable element 102. The bearing cup 1308 is restrained by a release 1310, which prevents the deployment spring 1306 from deploying the deployable element 102 until the release 1310 is moved. The bearing cup 1308 may include protrusions that allow the introducer 1318 to be withdrawn, as described in more detail below in connection with FIG. 15.

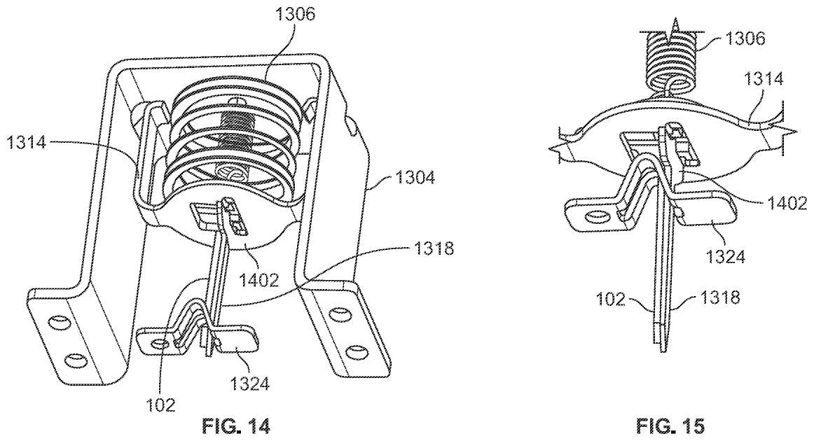

The buffer plate 1312 fits in or on a complimentary shaped opening in the suspension 1314 and supports the deployable element 102. The opening in the suspension 1314 has an opening with a first relatively narrow side and a second relatively wide side. The relatively narrow side allows the deployable element 102 and introducer 1318 to be deployed. The introducer 1318 is then pushed, due to the shape of the kick spring 1324, towards the second relatively wide side of the suspension 1314 and thereby retracts due to the action of the retraction spring 1316 (see description in connection with FIG. 14 and FIG. 15).

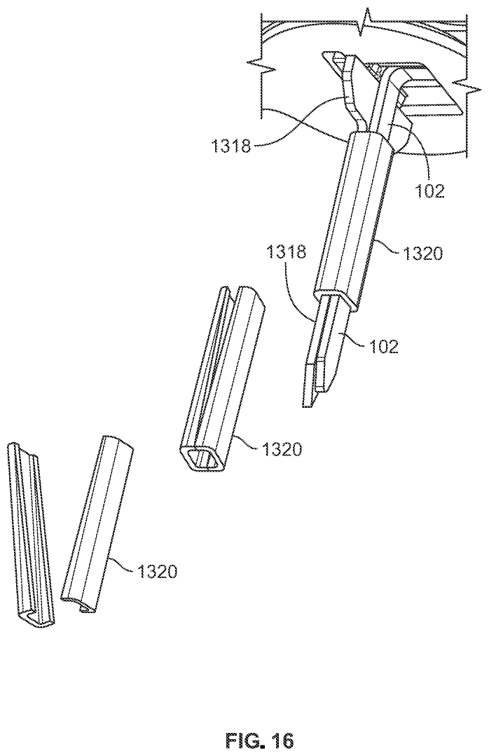

A sacrificial sleeve 1320 may temporarily hold the introducer 1318 and the deployable element 102 together, allowing them to be moved as a unit. See FIG. 16 for a more detailed description of the sacrificial sleeve 1320.

The kick spring 1324 and a support tube 1322 may be attached directly to the base 1326. The remaining elements of the conduit deployment assembly 1300 may attach to the harness 1304, which is then affixed to the base at the bottom of the harness 1304 legs.

In operation, as shown in FIG. 14, the deployment spring 1306 is initially held in a compressed state by the release 1310 so that it stores energy. When the release 1310 is removed, the energy stored in the deployment spring 1306 is released and transmitted to the bearing cup 1308. The bearing cup 1308 in turn pushes the deployable element 102 through the opening in the suspension 1314 alongside the introducer 1318, which may be joined to the deployable element 102. This causes the combination of the deployable element 102 and introducer 1318 to translate linearly for a first predetermined distance. This action causes the retraction spring 1316 attached to the introducer 1318 to extend, thus storing energy in the retraction spring 1316.

After the first predetermined distance, both the deployable element 102 and the introducer 1318 move through the opening in the kick spring 1324. The pointed end of the introducer 1318 creates an opening in the user's skin, through which the deployable element 102 can pass.

The opening in the kick spring 1324 and the introducer 1318 are shaped in a complementary manner so that, as the introducer 1318 moves down through the opening in the kick spring 1324, the introducer is pushed towards the side of the kick spring 1324 (see FIG. 15). The introducer 1318 includes a geometric portion having an angled structure 1402 that is wider than a first portion of an opening in the suspension 1314 through which the introducer 1318 initially passes. A second portion of the opening in the suspension 1314, which is oriented towards the same side of the suspension 1314 as the direction in which the kick spring 1324 pushes the introducer 1318, has a larger opening corresponding to the size and shape of the angled structure 1402.

As the angled structure 1402 moves toward the second portion of the opening due to the interaction of the kick spring 1324 on the introducer 1318, two things happen. First, the introducer 1318 moves towards the side of the bearing cup 1308, at which location a groove is cut into the bearing cup 1308. This releases the introducer 1318 from the action of the deployment spring 1306. Second, the retraction spring 1316, which was previously restrained from pulling back on the introducer 1318 by the suspension 1314, is free to pull the introducer 1318 back above the suspension 1314. This pulls the introducer 1318 back inside the base 1326, providing it with a secure housing that prevents the pointed end of the introducer 1318 from coming back into contact with the user's skin.

As noted above, the introducer 1318 and the deployable element 102 may be joined so that they move together linearly. FIG. 16 depicts an example of a sacrificial sleeve 1320 configured to temporarily join these two elements. The sacrificial sleeve 1320 temporarily increases the structural stiffness of the elements during combined translation in one desired direction, but subsequently slices, peels, crumples, or otherwise separates from the elements upon achieving a desired amount of translation or position. This allows separate slender elements to move disconnected and differentially relative to each other in the same or opposite directions.

For example, the sacrificial sleeve 1320 may be a thin-walled element formed of polymer material. As the suspension 1314 moves down under the action of the deployment spring 1306, the sacrificial sleeve 1320 may be compressed against the kick spring 1324. At this point, it may crumple, cut, or separate along a predefined separation line. The sacrificial sleeve 1320 may remain inside the conduit deployment assembly 1300, such as by being captured in the support tube 1322.

Similar to the embodiment described above, the deployable element 102 may be a cannula or needle for delivering a medicament such as insulin, or may be a glucose sensor or ketone sensor (e.g., an electrochemical sensor; a fiber optic sensor; a wire sensor; an optofluorescence sensor, etc.), a temperature sensor, or an activity sensor.

### Structures for Epidermal Tensioning

Because the above-described embodiments may be used with particularly thin, flexible elements, these elements may be especially susceptible to buckling or deflection. Any of these embodiments may benefit from deploying the cannulas or sensors (or the deployable element 102) into pre-tensioned tissue to reduce the chance of buckling. FIG. 17-FIG. 20 depict exemplary structures allowing the user's tissue (e.g., epidermal tissue) to be tensioned before the cannula/sensor is deployed.

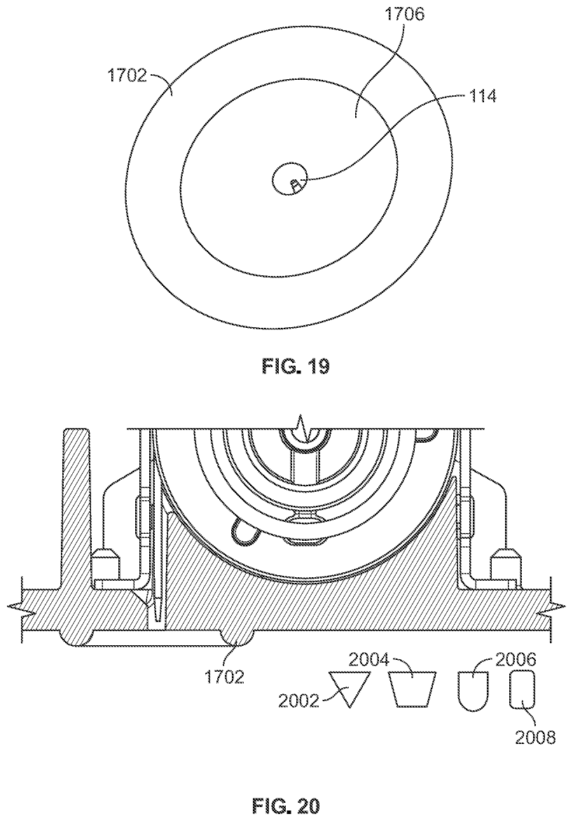

FIG. 17 and FIG. 19 depict an example of a tensioning structure that may be present on the base 108 of the conduit deployment assembly 100. The tensioning structure includes an annular ring 1702 centered on the opening in the base of the conduit deployment assembly 100 or conduit deployment assembly 1300 (through which the deployable element 102 passes). The annular ring 1702 may be of any suitable size; in the depicted example, the annular ring 1702 has an outer diameter of about 0.25 inches.

The annular ring 1702 creates a gap 1706 in the center of the annular ring 1702. As the base of the conduit deployment assembly is pressed into a user's skin 1704, the skin subtly bulges into the gap 1706, which tensions the skin 1704.

The example in FIG. 17 shows an annular ring having a semicircular cross-section. This, however, is not required, and other shapes may be suitable depending on the application. For example, FIG. 20 depicts cross sections for the annular ring having different shapes: a triangular cross-section 2002, a trapezoidal cross-section 2004, an ovoid cross-section 2006, and a rectangular cross section 2008.

FIG. 18 depicts an alternative configuration for the tensioning element. In this case, the tensioning element causes the skin 1704 to depress inwards, rather than bulging outwards as in FIG. 17. To achieve this, the bottom surface of the base 108 is projected outwards with a convex protrusion 1802 that may be centered on the opening 114. This pushes the skin 1704 away from the base 108, which causes it to pull tight against the base 108. If the deployable element 102 is being deployed at an angle, the opening 114 may be positioned such that the deployable element 102 and the tensioned skin surface where the deployable element 102 is to be inserted are nearly perpendicular to each other, or more perpendicular than they otherwise would be if the opening was at a center of the protrusion 1802.

### Exemplary Cannula and Sensor Structures

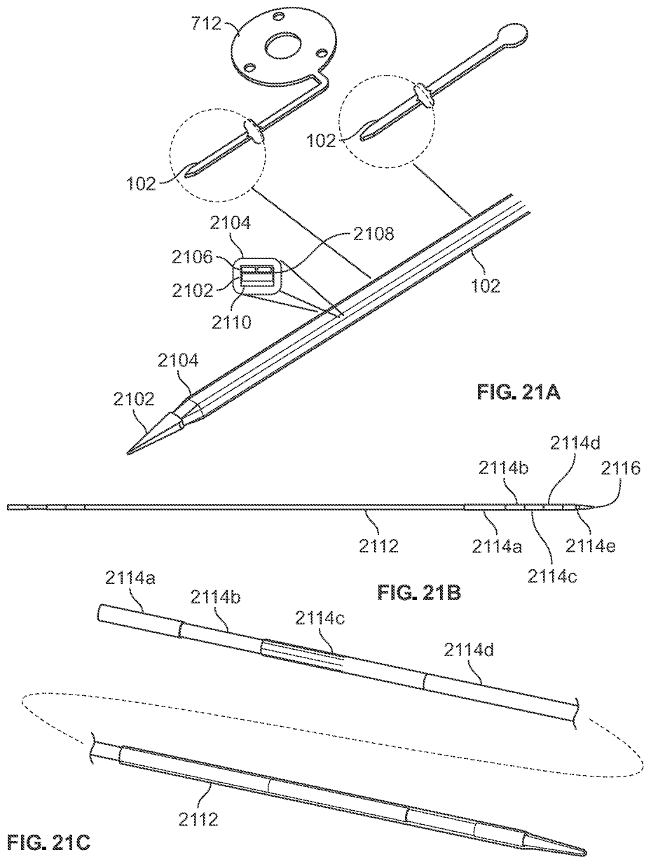

The cannula or sensor itself may also be structured or configured to support deployment, as shown in the example deployable elements 102 of FIG. 21A-FIG. 23.

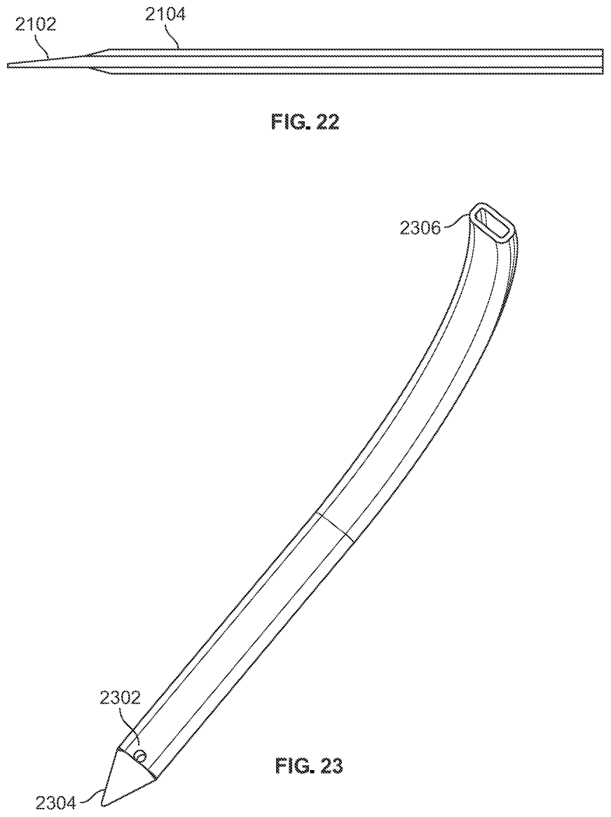

For instance, FIG. 21A-FIG. 22 depict an exemplary sensor made up of a laminated composition of polymers, metals, and other materials. These materials may be combined into varied cross-sections, and may be employed to develop desired directional stiffness (generally, although not necessarily, axial stiffness). This can improve deployment penetration of thin, flexible, and slender elements into human skin for a multi-day embedded use duration. Further it can maintain electrically conductive signal conduit/traces to embedded electronics hardware.

FIG. 21A depicts, at the top, two different deployable elements 102; the one on the left corresponds to the deployable element from the first, rotational embodiment, whereas the one on the right corresponds to the deployable element from the second, linear embodiment. The close-up at the bottom of the page represents one structure that could be suitable for use with either of these embodiments.

The deployable element 102 may be made up of a core layer 2102, an outer layer 2104, a first conductor/electrode 2106, a second conductor/electrode 2108, and/or a support 2110.

The core layer 2102 may be formed of a material that is sufficiently stiff or has a sufficiently high modulus to prevent the deployable element 102 from buckling or deflecting (e.g., stainless steel, titanium or another metal). A support 2110 may further improve the directional stiffness of the deployable element 102 for improving deployment penetration, and may be made from similar materials to the core layer 2102 and/or polymers, such as polyimides, FEP's, PTFE's, PP's, PCL's, PGA's, PGLA's, PLA's, silicone, etc. The support 2110 may also or alternatively be a biological specialty coating.

The outer layer 2104 may be a coating, such as enzymes or other bio-engineered compounds, which protects the other layers while protecting the penetration point into the user's skin. It may also be electrically insulative, thus preserving electrical signals traveling along the conductor/electrodes 2106, 2108.

The first and second conductor/electrodes 2106, 2108 may form traces that carry electrical signals from the distal end of the deployable element 102 to embedded circuitry used to process the signals. The conductor/electrodes 2106, 2108 may be made up, for example, of conductive material such as copper alloys, nickel, silver, gold, etc. The conductor/electrodes 2106, 2106 may extend along the entire length of the deployable element 102.

Once assembled, the multilayer construction may be modified at the distal tip to support penetration into the user's skin. For example, the material at the distal end could be ablated, thus removing all the material except for the core layer 2102 and allowing the core layer 2102 to be sharpened.

FIG. 21B and FIG. 21C depict additional examples of a deployable element in the form of a thin, flexible, multilayer laminated wire. In these embodiments, different layers 2114a 2114b, 2114c, 2114d, 2114e, . . . may be sequentially coated on top of a core 2116. For example, the core 2116 may be a length of wire (e.g., 5/1000s of an inch in diameter) made of tungsten or stainless steel. The layers 2114a, . . . May be insulators, conductors, enzymes, etc. coated down the length of the wire. A final layer may be a protective biocompatible coating 2112.

In some embodiments (e.g., shown in FIG. 21B), the different layers may be ablated away so that each is exposed in a sequence. Alternatively (FIG. 21C), various layers may be ablated in particular areas along the length of the wire to selectively expose them.

FIG. 23 depicts an exemplary structure suitable for a conduit. The conduit may include a strengthened tip 2304 at a distal end configured to penetrate the user's skin. The tip 2304 may be pointed or sharpened to facilitate penetration.

The conduit may be hollow to allow for a liquid drug to be dispensed through the conduit. To that end, one or more ports 2302 may be present at the distal end of the conduit in the vicinity of the tip 2304. The ports 2302 may face to the side(s) of the conduit to allow the liquid drug to be dispensed.

The conduit may, as in the depicted example, have a substantially rectangular cross section 2306. This cross section 2306 provides strength to the conduit and prevents it from collapsing, which would prevent the conduit from dispensing the liquid drug. Other suitable shapes may be used as appropriate. The proximal end of the conduit may include an interface allowing the conduit to be connected to a fluid conduit 202 that connects to a reservoir for supplying the liquid drug.

The rotary deployment mechanism depicted in FIGS. 1A-12D has been previously described as being “introducer-less.” Such a system is capable of deploying slender elements or deployable elements to depths of (for example) 2 to 10 millimeters as measured perpendicularly from the outer skin surface or, alternatively, measured linearly along the length of the deployable element, which “introducer-less” nature can help to minimize trauma and minimize wound size during penetration. Minimizing these factors is known to improve the efficacy of function of the slender element that resides within the tissue (generally at subcutaneous depth).

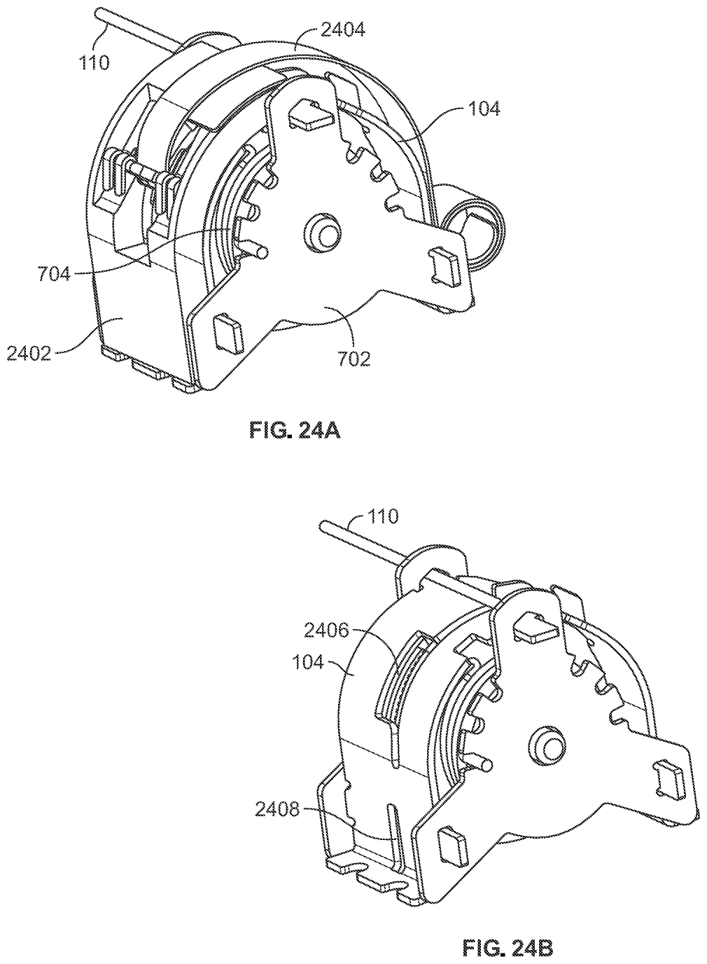

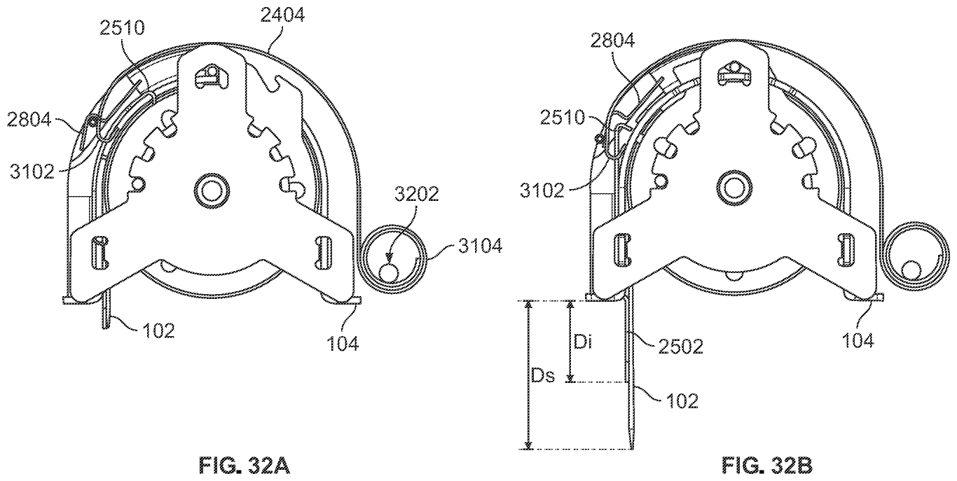

The embodiments depicted in FIGS. 24A-32B include introducers that are capable of minimally penetrating the toughest and strongest outermost layers of skin (stratum corneum through dermis), to a depth of only 1 to 3 millimeters, and then retract from the skin during or after the deployable element 102 (see, e.g., FIGS. 32A-32B) penetrates further down into the subcutaneous tissue. Therefore, the accompanying deployable element will only have to independently slide into softer subcutaneous layers or the least tough layers of the skin (mostly adipose and connective tissue) and remain there for the duration of use. Of course, an assisting system can be configured to drive the assisting sharp element to the full depth along with the deployable element; nonetheless, reduced penetration may be desirable for minimizing trauma and wound size.

These embodiments are particularly useful when applied in connection with slender elements or deployable elements that are so small in size or diameter, or have geometries or are made from particular materials, such that their intrinsic stiffness (e.g., Young's Modulus) renders them too weak to deliver independently into tissue media through skin without buckling.

The following figures include many of the same features as were previously addressed in FIGS. 1A-12D. Redundant descriptions are therefore omitted. FIG. 24A depicts an exemplary rotary cannula deployment mechanism that utilizes an introducer to penetrate the outermost layers of skin, while FIG. 24B depicts the rotary cannula deployment mechanism of FIG. 24A with an exemplary cowling 704.

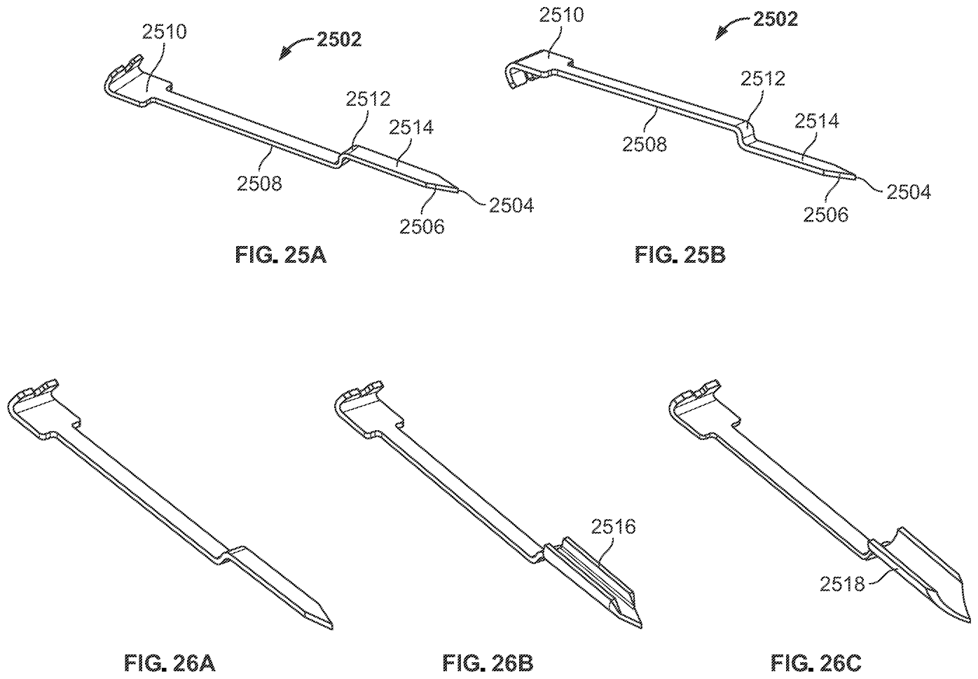

The cowling 704 is provided with integrated passages, including a first passage 2406 and a second passage 2408 in this embodiment. These passages are configured to fit, align, and guide an introducer (shown, for example, in FIGS. 25A-26C) that serves as the primary tissue penetrating element.

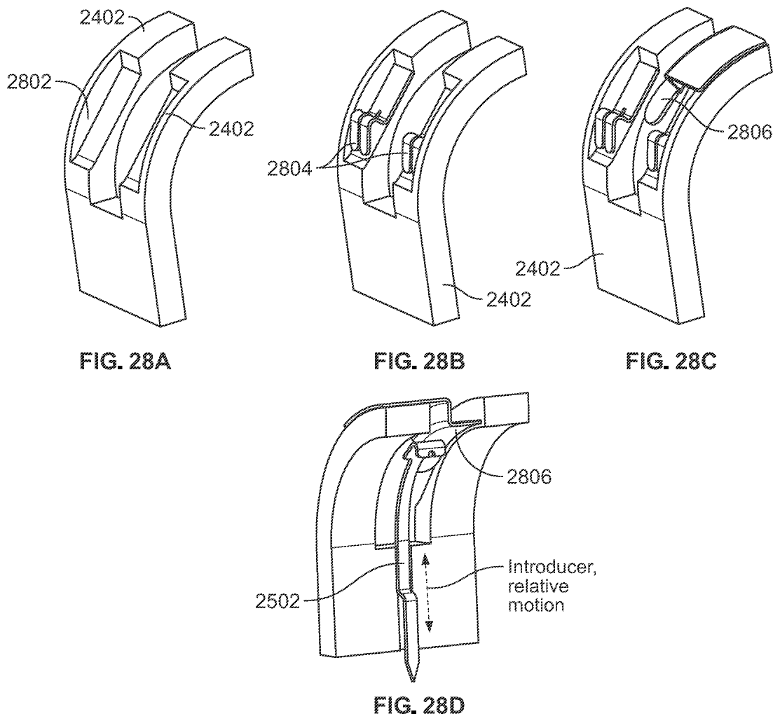

The deployment mechanism is also provided with a retaining block 2402 inside the cowling and an introducer spring 2404. The design and function of these elements is described in connection with FIGS. 28A-28D and 30A-31.

FIGS. 25A and 25B depict an exemplary introducer 2502 suitable for use with the described embodiments. The introducer 2502 may include a tapered, pointed end 2504 with sharpened edges 2506.

The introducer 2502 further includes a flexible portion 2508 that provides a spring-like portion allowing for longitudinal bending without yielding. When the introducer is in a pre-deployment configuration, the flexible portion 2508 curves around the cowling 104 and is driven by the above-described rotary motion of the sheave 706. As the introducer is deployed, the flexible portion 2508 straightens, driving the pointed end 2504 into the skin.

An interface feature 2510 such as a protrusion, recess, flange, or hook, allows the introducer 2502 to be connected to the introducer spring 2404, as shown in more detail in FIGS. 31A-31B.

An alignment feature 2512 may be a bend or curve that allows the portion of the introducer 2502 closest to the pointed end 2504, referred to herein as the introducer guide 2514, to be positioned proximate to or flush with the deployable element 102. Accordingly, the introducer can penetrate the outermost layers of the skin and guide the deployable element 102 into the body.

As shown in FIGS. 26A-26C, it is envisioned that some embodiments of an introducer 2502 may include various shapes and end conditions 2516, 2518 specifically configured for specialized applications. A few variants are depicted here as examples, but these are not all-inclusive of the many possible options.

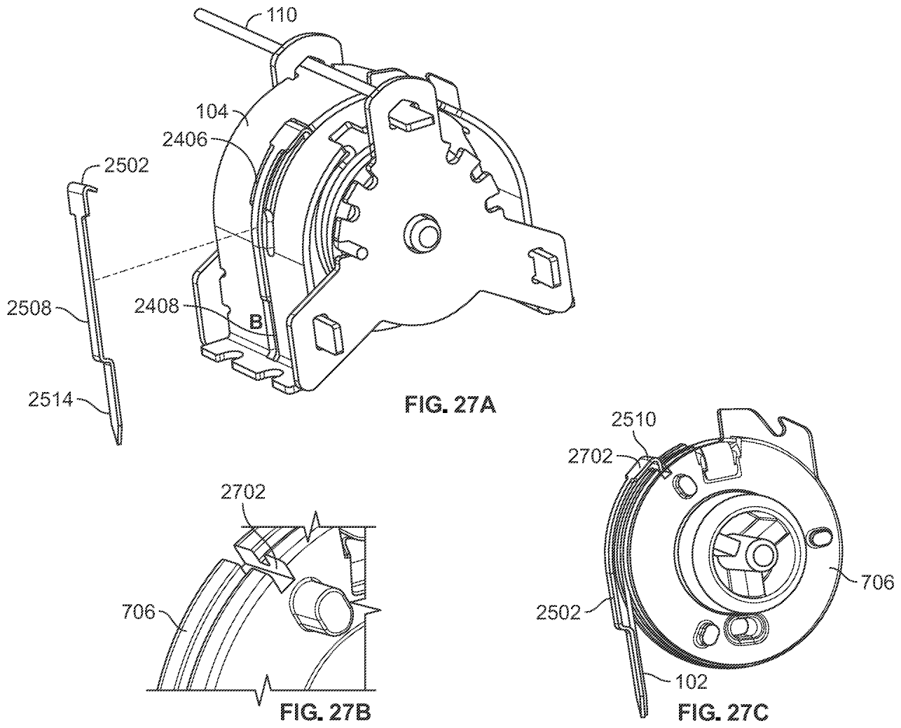

FIGS. 27A-C show how the exemplary introducer 2502 can be positioned with reference to the first passage 2406 and second passage 2408 of the cowling 104, and with the sheave 706. The interface feature 2510 of the introducer 2502 is configured to rest in a corresponding groove 2702 of the sheave 706. When assembled, the interface feature 2510 extends through the first passage 2406 of the cowling 104, while the flexible portion 2508 extends outside and around the cowling 104. The alignment feature 2512 causes the introducer 2502 to bend back towards the deployable element 102 and through the second passage 2408 of the cowling 104.

During assembly the flexible portion 2508 of the introducer 2502 is flexed to the geometry of the sheave 706 and the interface feature 2510 is fed into the groove 2702 on the sheave 706.

The major length of the introducer 2502 (corresponding to the flexible portion 2508) resides tangent to the outermost surface of the cowling 104 while a shorter length (corresponding to the introducer guide 2514), fits within the second passage 2408 integrated in the cowling 104.

This configuration also includes several other components which may be separate pieces combined by assembly. Alternatively or in addition, some elements may be integrated into single consolidated parts. FIGS. 28A-28D depict these additional components.

With the introducer 2502 located at its necessary position during assembly, a retaining block 2402 is added to secure the introducer 2502 with the cowling 104 and sheave 706. The retaining block 2492 may be, for example, an injection molded part of a polymeric material choice having low friction to reduce mechanical energy loss during functional motions of the relative sliding action of the introducer 2502. The retaining block 2402, along with any attached (or integrated) components, is directly affixed to the cowling 104 or another static element of the system by means of snap-fits, press-fits, crimps, or other suitable attachment methods.

The retaining block 2402 may include recesses 2802 configured and positioned to locate, constrain, and hold escapement clip(s) 2804, or similar elements, which provide low force flexing and serve to set the position of introducer spring 2402 and a latching element within the system. The introducer spring 2402 may provide sufficient force to retract the introducer 2502 during system function. A representative introducer spring 2402 is shown in more detail in FIGS. 31A-31B.

The escapement clips 2804 may be configured of stamped and formed thin metal parts. Such escapement clips 2804 could also become part of or affixed to the cowling 104. Likewise, a retaining flexure 2806 is included in the configuration. The retaining flexure's 2806 main function is to provide a light normal force, radially directed inward towards the cowling's 104 center, applied (e.g., directly) on the introducer's 2502 outward facing surface. This retaining flexure 2806 also establishes the initial escapement position that allows the introducer 2502 to snap and flex from its temporarily held curvature shape back to its original straightened beam shape. The retaining flexure 2806 in this embodiment is configured of stamped and formed thin metal with attachment features and a flexing cantilever beam-type segment of the geometry. This element could also attach directly to the cowling 104.

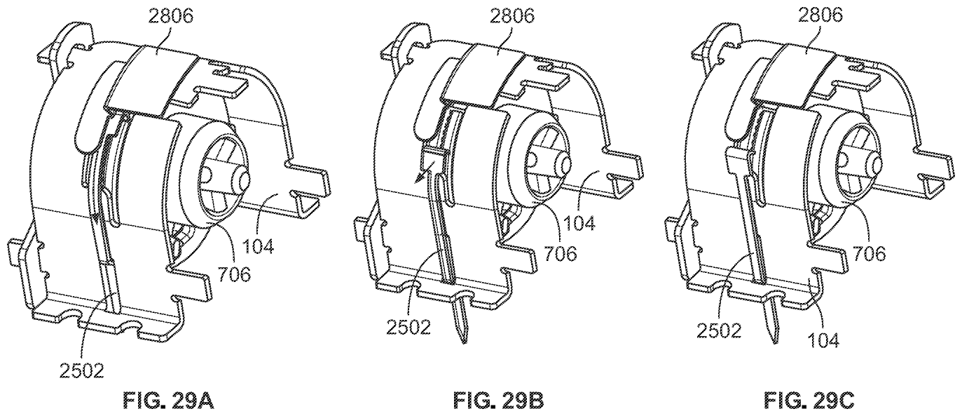

FIGS. 29A-C show the exemplary sequence of positions for the introducer 2502 during deployment and prior to retraction. FIG. 29A shows the initial position of the introducer 2502 at the onset of the rotary motion of the contacting sheave 706. FIG. 29B shows the position at which the introducer 2502 is freed from the retaining flexure 2806 constraining force and the sheave's 706 rotation. FIG. 29C shows the momentary state where the introducer 2502 is sprung back to its as manufactured straightened shape, and just prior to its retraction by the introducer spring 2404.

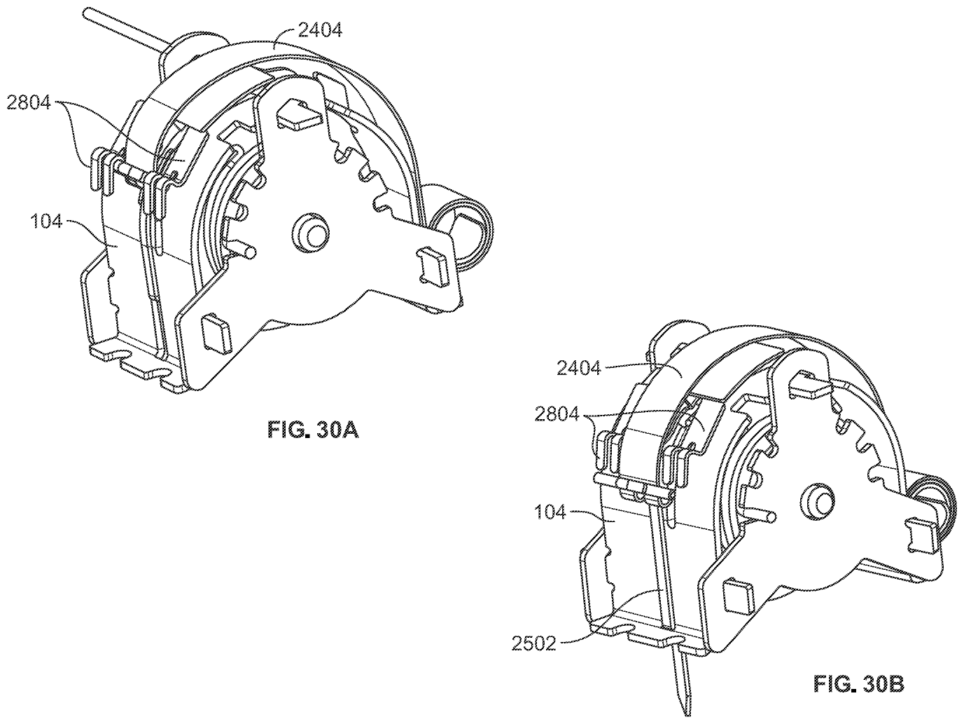

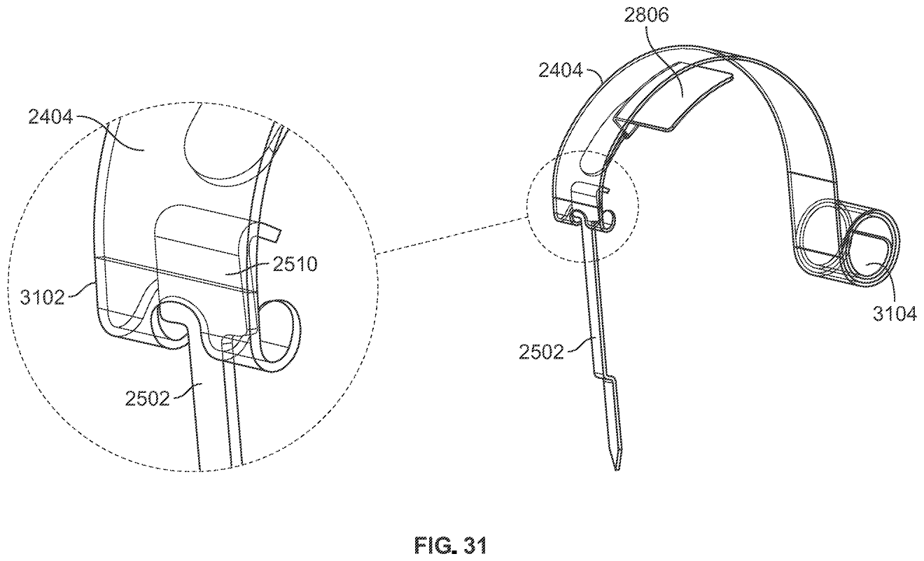

During its fully penetrated (deployed) position, the introducer 2502 is engaged with geometry features integrated within or assembled to the introducer spring 2404 through the interface feature 2510. FIG. 30B, which shows the introducer 2502 at the fully penetrated position, shows the point at which the introducer spring 2404, while now linked to the introducer 2502, is freed from its static holding position under the escapement clips 2804 and therefore allowed to retract the introducer 2502 from the penetrated tissue.

FIG. 30A, which shows the introducer 2502 in a pre-deployed state, shows that the load of the introducer spring 2404 (a constant force type in this embodiment) is held by the escapement clips 2804 and therefore isolated (or separated) from the main driving energy source during the major degree of the driving rotary motion. The system configuration helps to reduce the required force capability of the introducer spring 2404 such that it is only slightly greater than the friction forces on the introducer 2502 resulting from contact with tissue and the cowling 104 and retaining block 2402 surfaces. Therefore, this spring force would also minimally impede the main forces and inertia of the rotary motion of the sheave 706.

FIG. 31 is representative of exemplary interfacing features of the introducer 2502 and the interface spring 2404. These features allow these elements to be connected for retraction of the introducer 2502 during the rotary motion cycle of the system. The introducer interface feature 2510 has been addressed above. Meanwhile, a spring interface 3102 has a corresponding geometry, such as a hook for connecting to the hooked end of the introducer interface feature 2510 and a central hole through which the introducer 2502 passes through. In some embodiments, the end of the introducer spring 2404 can easily be formed into a subtle “U” or hook shape which will latch onto a flattened edge shape of the introducer interface feature 2510. This embodiment is representative of a most basic configuration for function. However, to those skilled with the art, alternative features that would provide for the interfacing functionality by varying nuances of the components and interfacing geometry and materials will be readily apparent.

The opposite end of the introducer spring 2404 may be shaped into a coil 3104, which can serve to reduce the overall size of the system.

FIGS. 32A-B correspond to FIGS. 30A and 30B, respectively, and show the comparison of the exemplary “Pre-Deployment State” to the state when the introducer 2502 and the deployable element 102 are at “Full Penetration” positions.

These Figures show that, prior to deployment, the introducer 2502 and the deployable element 102 tip locations are held at approximately the same distal position, though the introducer could extend distally beyond the deployable element 102 in an alternative embodiment. Upon a deployment triggering event, the introducer 2502 and deployable element 102 move in tangent proximity to each other at the same rate of excursion or penetration. Upon reaching its position of escapement during rotary motion, the introducer 2502 achieves its maximum penetrating depth (Di) within skin tissue, which as previously indicated may be 1 to 3 millimeters beneath the outer most surface of the skin. At this point in the rotation, the introducer 2502 is pulled upon by the introducer spring 2404 and retracts, traveling in the reverse direction and residing back inside the deployment device's enclosure where the introducer's 2502 tip stops and is contained at its initial position.

Meanwhile, the rotary motion of the sheave 706 continues along its forward direction, and the deployable element 102 (directly affixed to the sheave 706, at the deployable element's 102 opposite end from its tip in this embodiment) continues its trajectory into the tissue until its tip reaches a final depth (Ds) of, for example, 2 to 10 millimeters, or more preferably 4 to 6 millimeters, as measured perpendicularly from the outer skin surface or, alternatively, measured linearly along the length of the deployable element 102 (for example, when the angle of penetration is designed to not be perpendicular to the skin surface). When the rotary motion reaches its stop position, the deployable element 102 remains within the tissue for the duration of its use.

The figures also show a fixed point 3202 (shaft-like in this embodiment, although other configurations are also possible) that holds the coiled portion 3104 of the introducer spring 2404, allowing it to unwind and then pull-back. It is possible to configure similar functionality with other appropriately configured elastic, spring-like elements. It is also possible to adjust the angle of penetration from being perpendicular to, for example, being 30-60 degrees.

Having an introducer element that does not penetrate as far down into tissue as the deployable element 102 (e.g., an infusion cannula or a sensor element, as explained above), achieves many objectives. The introducer element is able to penetrate the tougher layers of skin (e.g., stratum corneum) to create a hole or introductory pathway for the more bendable deployable element 102, and then retract back into the delivering device (e.g., within a drug delivery device or a continuous glucose meter housing). This results in less pain to the user, and less trauma to the location where the penetration has occurred. Accordingly, less “wound healing” needs to occur with this design and approach. For a sensor, such as a continuous glucose sensor, for example, this reduced wound healing allows for the sensor to “warm up” more quickly and allows the sensor to provide readings, or more accurate readings, more quickly than is otherwise the case where an introducer or needle element penetrates to a depth more than or approximately equal to a depth that the sensor (or deployable element) penetrates. Less trauma is also beneficial when a cannula, for example, is the deployable element 102, and a drug, such as insulin or GLP-1 or similar drugs, are infused into the patient through the cannula. Reduced trauma and less wound healing can also reduce the likelihood of occlusion at or near the distal end of the cannula. Thus, having an introducer (which does not remain below the skin surface) that does not penetrate as far as the deployable element (which does remain below the skin surface) is highly beneficial for both the patient and the functionality of the medical device (whether a drug infusion device or a continuous glucose monitor (CGM), for example).

One of ordinary skill in the art would appreciate that the above concepts can be applied to a needle, which acts as an introducer, and an infusion cannula or an analyte sensor, which acts as a deployable element, as described above. More specifically, the introducer described above could be in the form of a needle, in some embodiments. The needle could be positioned inside the lumen of a hollow deployable element. In other embodiments, the needle could be hollow and the deployable element could be positioned inside the lumen of the needle. And as described above, the needle could penetrate through the outermost (toughest) layers of skin and then retract back out of the skin into the medical device housing where the needle was deployed from, and the deployable element can continue to penetrate further down into the softer layers of subcutaneous tissue (either while the needle is retracting backward or before the needle retracts backward, but after the needle stops penetrating farther distally). In this manner, the needle does not penetrate farther distally than the deployable element, leading to less pain for the user and less trauma at the insertion site.

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

Exemplary embodiments may include, but are not limited to:

1. An apparatus comprising: a deployable element configured to be deployed subcutaneously; an introducer configured so as to remain adjacent to the deployable element during a deployment of the deployable element to support the deployable element during deployment; and a base having an opening through which the deployable element and introducer are configured to extend, wherein the deployable element is configured to extend into a user's skin by a first amount during the deployment, and the introducer is configured so that the introducer penetrates and extends into the user's skin by a second amount that is less than the first amount.

2. An apparatus as described above in (1), wherein the deployable element is configured to extend into the user's skin to a depth of 4-7 mm.

3. An apparatus as described above in (1) or (2), wherein the introducer is configured to penetrate outermost layers of the user's skin comprising stratum corneum through dermis layers.

4. An apparatus as described above in any of (1)-(3), wherein the introducer is configured to extend into the user's skin to a depth of 1-3 mm.

5. An apparatus as described above in any of (1)-(4), wherein the introducer is configured to automatically retract through the opening in the base after reaching a maximum deployment distance.

6. An apparatus as described above in any of (1)-(5), wherein the deployable element is configured to continue to extend after the introducer reaches a maximum deployment distance.

7. An apparatus as described above in any of (1)-(6), wherein the deployable element is selected from the group consisting of a cannula, a conduit, a needle, or a sensor.

8. An apparatus as described above in any of (1)-(7), wherein the deployable element is: a glucose sensor configured to perform continuous glucose monitoring, or a ketone sensor.

9. An apparatus as described above in any of (1)-(8), further comprising a rotary mechanism having an outer circumference around at least a portion of which the deployable element is wrapped, the rotary mechanism configured to rotate the deployable element around the outer circumference and configured to selectively constrain the deployable element so that the rotation of the deployable element is converted into a linear motion.

10. An apparatus as described above in (9), further comprising an introducer spring that is coiled around a fixed point on the apparatus.

11. An apparatus as described above in (10), further comprising: a cowling enclosing at least a portion of the rotary mechanism; a retaining block configured to secure the introducer to the cowling; and one or more escapement clips provided in corresponding recesses of the retaining block and configured to secure the introducer spring.

12. An apparatus as described above in any of (10)-(11), wherein the introducer spring comprises an introducer spring interface configured to mate with a corresponding interface on the introducer.

13. An apparatus as described above in any of (9)-(12), wherein the rotary mechanism is a sheave assembly.

14. An apparatus as described above in any of (9)-(13), further comprising a torsion spring configured to supply energy to rotate the rotary mechanism.

15. An apparatus as described above in any of (9)-(14), further comprising a stop plate configured to control an extent to which the deployable element extends beyond the opening.

16. An apparatus as described above in any of (9)-(15), further comprising a fluid conduit configured to connect the deployable element to a reservoir.

17. An apparatus as described above in any of (1)-(8), further comprising a linear deployment mechanism configured to cause the deployable element and the introducer to extend.

18. An apparatus as described above in any of (1)-(17), further comprising at least one convex or concave protrusion provided on the base, wherein the protrusion is sized and shaped to cause a user's skin to be tensioned in the vicinity of the opening when the base is pushed against the skin.

19. An apparatus as described above in any of (1)-(18), wherein the deployable element has a laminated composition of comprising at least one polymer, at least one metal, and a coating.

20. An apparatus as described above in any of (1)-(19), wherein the deployable element comprises one or more electrically conductive signal traces connected to embedded electronics hardware.

What is claimed is:

1. An apparatus comprising:
a deployable element configured to be deployed subcutaneously;
an introducer configured so as to remain adjacent to the deployable element during a deployment of the deployable element to support the deployable element during deployment; and
a base having an opening through which the deployable element and introducer are configured to extend,
wherein the deployable element is configured to extend into a user's skin by a first amount during the deployment, and the introducer is configured so that the introducer penetrates and extends into the user's skin by a second amount that is less than the first amount.

2. The apparatus of claim 1, wherein the deployable element is configured to extend into the user's skin to a depth of 4-7 mm.

3. The apparatus of claim 1, wherein the introducer is configured to penetrate outermost layers of the user's skin comprising stratum corneum through dermis layers.

4. The apparatus of claim 1, wherein the introducer is configured to extend into the user's skin to a depth of 1-3 mm.

5. The apparatus of claim 1, wherein the introducer is configured to automatically retract through the opening in the base after reaching a maximum deployment distance.

6. The apparatus of claim 1, wherein the deployable element is configured to continue to extend after the introducer reaches a maximum deployment distance.

7. The apparatus of claim 1, wherein the deployable element is selected from the group consisting of a cannula, a conduit, a needle, or a sensor.

8. The apparatus of claim 1, wherein the deployable element is:
a glucose sensor configured to perform continuous glucose monitoring, or
a ketone sensor.

9. The apparatus of claim 1, further comprising a rotary mechanism having an outer circumference around at least a portion of which the deployable element is wrapped, the rotary mechanism configured to rotate the deployable element around the outer circumference and configured to selectively constrain the deployable element so that the rotation of the deployable element is converted into a linear motion.

10. The apparatus of claim 9, further comprising an introducer spring that is coiled around a fixed point on the apparatus.

11. The apparatus of claim 10, further comprising:
a cowling enclosing at least a portion of the rotary mechanism;
a retaining block configured to secure the introducer to the cowling; and
one or more escapement clips provided in corresponding recesses of the retaining block and configured to secure the introducer spring.

12. The apparatus of claim 10, wherein the introducer spring comprises an introducer spring interface configured to mate with a corresponding interface on the introducer.

13. The apparatus of claim 9, wherein the rotary mechanism is a sheave assembly.

14. The apparatus of claim 9, further comprising a torsion spring configured to supply energy to rotate the rotary mechanism.

15. The apparatus of claim 9, further comprising a stop plate configured to control an extent to which the deployable element extends beyond the opening.

16. The apparatus of claim 9, further comprising a fluid conduit configured to connect the deployable element to a reservoir.

17. The apparatus of claim 1, further comprising a linear deployment mechanism configured to cause the deployable element and the introducer to extend.

18. The apparatus of claim 1, further comprising at least one convex or concave protrusion provided on the base, wherein the protrusion is sized and shaped to cause a user's skin to be tensioned in the vicinity of the opening when the base is pushed against the skin.

19. The apparatus of claim 1, wherein the deployable element has a laminated composition of comprising at least one polymer, at least one metal, and a coating.

20. The apparatus of claim 1, wherein the deployable element comprises one or more electrically conductive signal traces connected to embedded electronics hardware.
